(19) United States
(12) Patent Application Publication
(10) Pub. No.: US 2019/0263796 A1
(43) Pub. Date: Aug. 29, 2019

(54) LIM KINASE INHIBITORS, PHARMACEUTICAL COMPOSITION AND METHOD OF USE IN LIMK-MEDIATED DISEASES

(71) Applicant: CELLIPSE, Saint-Cyr-au-Mont-d'Or (FR)

(72) Inventors: Renaud PRUDENT, Renage (FR); Fabrice PAUBLANT, Saint-Cyr-au-Mont-d'Or (FR)

(73) Assignee: CELLIPSE, Saint-Cyr-au-Mont-d'Or (FR)

(21) Appl. No.: 16/334,932

(22) PCT Filed: Sep. 22, 2017
(86) PCT No.: PCT/EP2017/074071

§ 371 Date: Mar. 20, 2019

(30) Foreign Application Priority Data
Sep. 23, 2016 (EP) 16190421.4
Sep. 23, 2016 (EP) 16190422.2

(51) Int. Cl. C07D 417/04; C07D 487/04; A61P 35/02

(52) U.S. Cl. CPC C07D 417/04; A61P 35/02; C07D 487/04

(57) ABSTRACT

The LIM Kinase inhibitors of Formula I

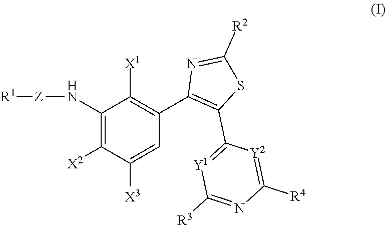

and pharmaceutically acceptable salts or solvates thereof, wherein R¹, R², R³, R⁴, X¹, X², X³, Y¹, Y² and Z are as defined in the claims. Also, the use of LIM Kinase inhibitors of Formula I for the treatment and/or prevention of LIMK-mediated diseases.

### FIELD OF INVENTION

The present invention relates to kinase inhibitors, more specifically LIM kinase (LIMK) inhibitors, to pharmaceutical compositions comprising such inhibitors, and to uses of such inhibitors in the treatment and/or prevention of LIMK-mediated diseases including proliferative conditions such as cancer and more specifically acute myeloid leukemia.

### BACKGROUND OF INVENTION

The LIM kinase family consists of two members: LIM kinase 1 (LIMK 1) and LIM kinase 2 (LIMK 2).

LIM kinases are regulated by several upstream signaling pathways, principally acting downstream of Rho GTPases (Scott and Olson, J. Mol. Med, 2007, 85, 555-568). Similar to many other kinases, phosphorylation in the activation loop results in increased LIMK activity. Both LIMK 1 and LIMK 2 are phosphorylated by the Rho effector Rho kinase (ROCK). Pak1, Pak2, Pak4 and the myotonic dystrophy kinase-related Cdc42-binding kinase (MRCKα) have also been each reported to phosphorylate and activate LIMK1 and/or LIMK2.

The main substrates of LIMK are cofilin 1, cofilin 2 and destrin, often generally referred to as “cofilin”.

LIM kinases influence the architecture of the actin cytoskeleton by regulating the activity of the cofilin proteins. Especially, LIM kinases act by phosphorylating cofilin and thereby inactivating its actin-severing activity, altering the rate of actin depolymerization and barbed end formation. Therefore, LIM kinases play a major role in the regulation of cells morphology and motility.

Through this modulation of the actin skeleton, LIMK is implicated in several conditions such as Williams syndrome, Alzheimer's disease, Parkinson's disease, intracranial aneurism, pulmonary hypertension, glaucoma, cardiovascular disorders or proliferative diseases such as cancer and metastasis (Scott and Olson, J. Mol. Med, 2007, 85, 555-568; Manetti, Current Cancer Drug Targets, 2012, 12, 543-560). Especially, perturbations in the balance between phosphorylated and non-phosphorylated cofilin is a significant determinant of tumor-cell invasion and metastasis and LIMK plays a central role therein, especially in solid tumors.

Acute myeloid leukemia (AML) is a heterogeneous hematologic malignancy. Therapeutic intervention for AML is largely ineffective and new treatments are expected. Recent reports from proteomic analysis have shown an unexpected link between AML and actin cytoskeleton components (cofilin, actin, moesin, ezrin, PAK1) (Jiang et al., J. Proteomics, 2011, 74(6), 843-857; Luczak et al., J. Proteomics, 2012, 75(18), 5734-5748; Braoudaki et al., Amino Acids, 2011, 40(3), 943-951; Habif et al., J. Proteomics, 2013, 78, 231-244). In line with, mutations frequently found in AML of tyrosine kinase receptors and transcription factors alter actin cytoskeleton dynamics (Tanaka et al., Oncogene, 1998, 17(6), 699-708; Mali et al., Cancer Cell, 2011, 20, 357-369). Especially, it was shown that targeting key components (PKCzeta, FAK) of actin regulation lead to antiproliferative effect of AML cells (Guo et al., J. Neurochem., 2009, 109(1), 203-213; Despeaux et al., Stem Cells, 2012, 30(8), 1597-1610). Rho GTPase/ROCK pathway is major modulator of actin dynamics and targeting this pathway in KIT, FLT3 or BCR-Abl mutated AML cells elicits selective anti-leukemic effect (Mali et al., Cancer Cell, 2011, 20, 357-369). Targeting Rho GTPase pathway thus appears as an attractive opportunity for new AML treatment (Kuzelova et al., Cardiovasc. Hematol. Disord. Drug Targets, 2008, 8(4), 261-267; Rath et al., EMBO Reports, 2012, 13(10), 900-908). LIM kinases are the last kinases involved in the Rho GTPase pathway. Several reports suggest that targeting LIMK or its substrate, cofilin (Guo et al., J. Neurochem., 2009, 109(1), 203-213; Nakashima et al., Bioorg. Med. Chem. Lett., 2010, 20(9), 2994-2997) in leukemia may be of therapeutic value. Especially, it has been evidenced that inhibiting LIMK exerts an anti-leukemic activity in murine model of leukemia (Prudent et al., Cancer Research, 2012, 72(17), 4429-4439).

Small molecules were proposed as LIMK inhibitors to treat various LIMK-related diseases (see for example WO2015/025172; WO2015/150337; WO2014/002101; WO2011/091204; WO2006/084017; Prudent et al., Cancer Research, 2012, 72(17), 4429-4439; Manetti, Med. Res. Rev., 2012, 32(5), 968-998).

However, despite several ongoing clinical trials in AML using drugs targeting upstream components (mainly tyrosine receptor kinase (FLT3, BCR-Abl, etc.) or receptor (CXCR4 (Foran et al., Hematology, 2012, Suppl 1, S137-140)) of LIMK, so far, no agent targeting LIM kinase is available to treat AML.

Therefore, there is a need for new LIMK inhibitors to treat LIMK-related diseases and more specifically to treat AML.

The Applicant herein provides compounds of Formula I as defined below, which are evidenced in the experimental part to be potent LIMK inhibitors.

### SUMMARY

This invention thus relates to a compound of Formula I:

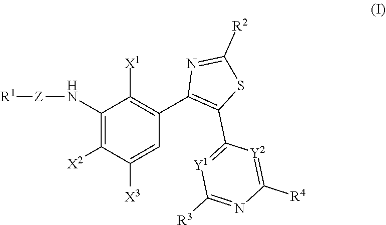

or a pharmaceutically acceptable salt or solvate thereof, wherein R¹, R², R³, R⁴, X¹, X², X³, Y¹, Y² and Z are as defined below.

According to one embodiment, the compound according of the invention is of Formula Ia, Ib, Ic, Id or Ie as defined below. According to one embodiment, the compound of the invention is of Formula Ia-U0, Ia-U1a, Ia-U1b, Ia-U3a, Ia-U3b or Ia-U8 as defined below. According to one embodiment, the compound of the invention is of Formula Ia-U0-1 as defined below. According to one embodiment, the compound of the invention is selected from the group consisting of:
N-(3-(2-(tert-butyl)-5-(2-(phenylamino)pyrimidin-4-yl)thiazol-4-yl)-2-fluorophenyl)-2,6-difluorobenzenesulfonamide;
ethyl 2-(4-(3-(2,6-difluorophenylsulfonamido)-2-fluorophenyl)-5-(2-(phenylamino)pyrimidin-4-yl)thiazol-2-yl)acetate;
N-(3-(2-(tert-butyl)-5-(2-(phenylamino)pyrimidin-4-yl)thiazol-4-yl)-2-fluorophenyl)propane-1-sulfonamide;
N-(3-(2-(tert-butyl)-5-(2-(phenylamino)pyrimidin-4-yl)thiazol-4-yl)-2-fluorophenyl)-3,3,3-trifluoropropane-1-sulfonamide;
N-(3-(2-(tert-butyl)-5-(2-(phenylamino)pyrimidin-4-yl)thiazol-4-yl)-2-fluorophenyl)-2-(2,6-difluorophenyl)-2-oxoacetamide;
tert-butyl (3-(2-(tert-butyl)-5-(2-(phenylamino)pyrimidin-4-yl)thiazol-4-yl)-2-fluorophenyl)carbamate;
N-(3-(2-(tert-butyl)-5-(7H-pyrrolo[2,3-d]pyrimidin-4-yl)thiazol-4-yl)-2-fluorophenyl)-2,6-difluorobenzenesulfonamide;
2,6-difluoro-N-(2-fluoro-3-(2-(2-hydroxyethyl)-5-(2-(phenylamino)pyrimidin-4-yl)thiazol-4-yl)phenyl)benzenesulfonamide;
2-(4-(3-(2,6-difluorophenylsulfonamido)-2-fluorophenyl)-5-(2-(phenylamino)pyrimidin-4-yl)thiazol-2-yl)-N-methylacetamide;
N-(3-(2-(tert-butyl)-5-(2-(phenylamino)pyrimidin-4-yl)thiazol-4-yl)-2-fluorophenyl)cyclopropanesulfonamide;
   and pharmaceutically acceptable salts or solvates thereof.

The invention also relates to a pharmaceutical composition comprising a compound according to the invention, or a pharmaceutically acceptable salt or solvate thereof, and at least one pharmaceutically acceptable excipient.

The invention also relates to a medicament comprising a compound according to the invention, or a pharmaceutically acceptable salt or solvate thereof.

The invention has also for objection a compound according to the invention, or a pharmaceutically acceptable salt or solvate thereof, for use in the treatment and/or the prevention of a LIMK-related disease.

According to one embodiment, the LIMK-related disease is selected from proliferative conditions, neurodegenerative disorders, neurodevelopmental disorders, cardiovascular and vascular diseases, eye diseases, airway diseases, inflammatory diseases, skin diseases, intestinal diseases, kidney diseases, bone diseases, viral diseases, drug addiction and neurofibromatosis.

According to one embodiment, the proliferative conditions are selected from tumors, cancers, neoplasms, hyperplasias, psoriasis, bone diseases, fibroproliferative disorders, pulmonary fibrosis, atherosclerosis and smooth muscle cell proliferation in the blood vessels.

According to one embodiment, the proliferative condition is selected from:
carcinomas, such as for example a carcinoma of the bladder, breast, colon, bowel, rectum, kidney, epidermal, liver, lung, esophagus, gall bladder, ovary, uterus, endometrium, pancreas, stomach, cervix, thyroid, prostate, testicle, skin, brain, nerve, bone;
hematopoietic tumors of lymphoid lineage, such as for example leukemia, acute lymphocytic leukemia, B-cell lymphoma, T-cell lymphoma, Hodgkin's lymphoma, non-Hodgkin's lymphoma, hairy cell lymphoma, or Burkett's lymphoma;
hematopoietic tumors of myeloid lineage, such as for example acute myeloid leukemia (including acute promyelocytic leukemia), chronic myeloid leukemia or myelodysplasia syndrome;
tumors of mesenchymal origin, such as for example fibrosarcoma or rhabdomyosarcoma;
tumors of the central or peripheral nervous system, such as for example astrocytoma, neuroblastoma, glioma or schwannoma;
melanoma; seminoma; teratocarcinoma; osteosarcoma; xeroderma pigmentosum;
keratoacanthoma; thyroid follicular cancer; or Kaposi's sarcoma.

According to one embodiment, the LIMK-related disease is acute myeloid leukemia.

The invention further relates to a process of manufacturing a compound according to the invention, or a pharmaceutically acceptable salt or solvate thereof, characterized in that it comprises the following steps:

a) reacting intermediate (A)

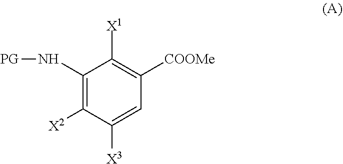

wherein PG represents an amino-protecting group; and wherein X¹, X², and X³ are as defined in Formula I;
   with intermediate (B)

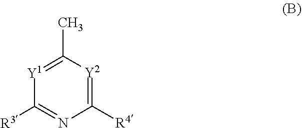

wherein Y¹ and Y² are as defined in Formula I; and
wherein R^{3′} and R^{4′} independently either represent respectively R³ or R⁴ as defined in in Formula I, or a precursor of respectively R³ or R⁴;
   in presence of a strong base, to afford intermediate (C)

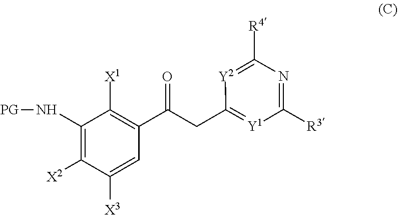

b) forming a thiazole ring by reacting intermediate (C) in presence of N-bromosuccinimide and intermediate (D)

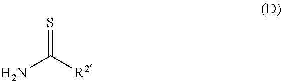

wherein R^{2′} either represents R² as defined in Formula I, or a precursor of R²; to afford intermediate E

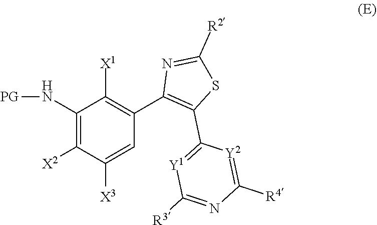

c) deprotecting intermediate (E) in conditions adapted to remove PG, to afford intermediate (F)

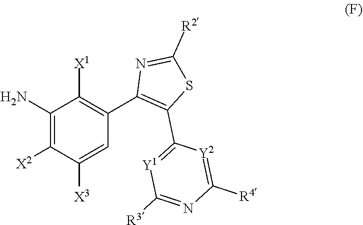

d) introducing R¹ moiety as defined in Formula I on intermediate (F) by suitable coupling reaction adapted to —Z— linker as defined in Formula I to afford compound of Formula I′

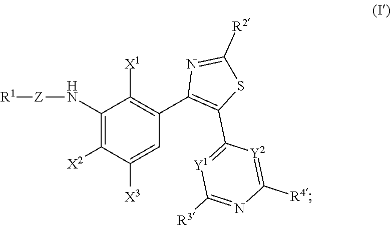

and in case wherein R^{2′}, R^{3′} and/or R^{4′} represent precursors of respectively R², R³ or R⁴, performing one or more additional intermediate steps or final steps of conversion of R^{2′} into R² and/or R^{3′} into R³ and/or of R^{4′} into R⁴.

### Definitions

In the present invention, the following terms have the following meanings:
“alkoxy” refers to a group —O-alkyl, wherein alkyl is as defined below. Suitable alkoxy groups include for example methoxy, ethoxy, n-propoxy, isopropoxy, n-butoxy, t-butoxy, sec-butoxy, and n-pentoxy.
“alkoxyalkyl” refers to a group -alkyl-O-alkyl, wherein alkyl is as defined below.
“alkyl” refers to a hydrocarbyl radical of formula CₙH₂ₙ₊₁ wherein n is a number greater than or equal to 1. Generally, alkyl groups of this invention comprise from 1 to 6 carbon atoms, preferably from 1 to 4 carbon atoms. Alkyl groups may be linear or branched. Suitable alkyl groups include but are not limited to methyl, ethyl, n-propyl, i-propyl, n-butyl, i-butyl, s-butyl, t-butyl, pentyl and its isomers (e.g. n-pentyl, i-pentyl), and hexyl and its isomers (e.g. n-hexyl, i-hexyl).
“alkylaminocarbonylalkyl” refers to a group -alkyl-CO—NH-alkyl, wherein alkyl is as define above. An example of such group is —CH₂—CONHMe.
“alkyloxycarbonylalkyl” refers to a group -alkyl-CO—O-alkyl, wherein alkyl is as define above. An example of such group is —CH₂—COOEt.
“aminoalkyl” refers to a group -alkyl-NH₂, wherein alkyl is as define above.
“aminocarbonylalkyl” refers to a group -alkyl-CO—NH₂, wherein alkyl is as define above. An example of such group is —CH₂—CONH₂.
“aryl” refers to a polyunsaturated, aromatic hydrocarbyl group having a single ring (i.e. phenyl) or multiple aromatic rings fused together (e.g. naphtyl) or linked covalently, typically containing 5 to 12 atoms; preferably 6 to 10, wherein at least one ring is aromatic. The aromatic ring may optionally include one to two additional rings (either cycloalkyl, heterocyclyl or heteroaryl) fused thereto. Aryl is also intended to include the partially hydrogenated derivatives of the carbocyclic systems enumerated herein. Non-limiting examples of aryl comprise phenyl, biphenylyl, biphenylenyl, 5- or 6-tetralinyl, naphthalen-1- or -2-yl, 4-, 5-, 6 or 7-indenyl, 1-2-, 3-, 4- or 5-acenaphtylenyl, 3-, 4- or 5-acenaphtenyl, 1- or 2-pentalenyl, 4- or 5-indanyl, 5-, 6-, 7- or 8-tetrahydronaphthyl, 1,2,3,4-tetrahydronaphthyl, 1,4-dihydronaphthyl, 1-, 2-, 3-, 4- or 5-pyrenyl.
“arylalkyl” refers to a group -alkyl-aryl, wherein alkyl and aryl are as herein defined.
“cycloalkyl” refers to a cyclic alkyl group, that is to say, a monovalent, saturated hydrocarbyl group having one cyclic structures. Cycloalkyl groups may comprise 3 or more carbon atoms in the ring and generally, according to this invention comprise from 3 to 10, more preferably from 3 to 8 carbon atoms still more preferably from 3 to 6 carbon atoms. Examples of cycloalkyl groups include but are not limited to cyclopropyl, cyclobutyl, cyclopentyl, cyclohexyl.
“halo” refers to fluoro, chloro, bromo, or iodo. Preferred halo groups are fluoro and chloro.
“haloalkyl” refers to any alkyl group substituted by one or more halo group. Non-limiting examples of haloalkyl groups are CF₃, CHF₂ and CH₂F.
“heteroaryl” refers to 5 to 12 carbon-atom aromatic rings or ring systems containing 1 to 2 rings which are fused together or linked covalently, typically containing 5 to 6 atoms; at least one of which is aromatic; in which one or more carbon atoms in one or more of these rings can be replaced by oxygen, nitrogen or sulfur atoms, where the nitrogen and sulfur heteroatoms may optionally be oxidized and the nitrogen heteroatoms may optionally be quaternized. Such rings typically comprise 1 to 4, preferably 1 or 2, heteroatoms per ring. Such rings may be fused to an aryl, cycloalkyl, heteroaryl or heterocyclyl ring. Non-limiting examples of such heteroaryl, include: pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, thienopyridinyl, purinyl, imidazo[1,2-a]pyridinyl, 6-oxo-pyridazin-1 (6H)-yl, 2-oxopyridin-1 (2H)-yl, 6-oxo-pyrudazin-1 (6H)-yl, 2-oxopyridin-1(2H)-yl, 1,3-benzodioxolyl, quinolinyl, isoquinolinyl, cinnolinyl, quinazolinyl, quinoxalinyl.
“hydroxyalkyl” refers to a group -alkyl-OH, wherein alkyl is as defined above.
“oxacycloalkyl” refers to a cycloalkyl group wherein one or more carbon atoms are exchanged for an oxygen atom. Non-limiting examples of such oxacycloalkyl include oxacyclopropanyl (ethylene oxide), oxacyclopentanyl (tetrahydrofuryl), oxacyclohexanyl (tetrahydropyranyl).
“patient” refers to a warm-blooded animal, more preferably a human, who/which is awaiting the receipt of, or is receiving medical care or is/will be the object of a medical procedure. The term “human” refers to a subject of both genders and at any stage of development (i.e. neonate, infant, juvenile, adolescent, adult (including young adult, middle-aged adult and older adult)).
“solvate” refers to a compound in this invention that contains stoichiometric or sub-stoichiometric amounts of one or more pharmaceutically acceptable solvent molecule such as ethanol. The term “hydrate” refers to when the said solvent is water.
“treat”, “treating” and “treatment” refer to therapeutic treatment, prophylactic or preventative measures and deferment of the disease onset; wherein the object is to delay, prevent or slow down (lessen) the targeted pathologic condition or disorder. Those in need of treatment include those already with a LIMK-related disease, as well as those prone to have a LIMK-related disease, or those in whom a LIMK-related disease is to be prevented or delayed. Parameters for assessing successful treatment and improvement in the disease are readily measurable by routine procedures familiar to the skilled artisan.
“prevent”, “preventing” and “prevention” refer to a method of delaying or precluding the onset of a condition or disease and/or its attendant symptoms, barring a patient from acquiring a condition or disease, or reducing a patient's risk of acquiring a condition or disease.
“therapeutically effective amount” (or more simply an “effective amount”) means the amount of active agent or active ingredient (e.g. LIMK antagonist) that is sufficient to achieve the desired therapeutic or prophylactic effect in the patient to which/whom it is administered.
“administration”, or a variant thereof (e.g. “administering”), means providing the active agent or active ingredient (e.g. a LIMK antagonist), alone or as part of a pharmaceutically acceptable composition, to the patient in whom/which the condition, symptom, or disease is to be treated or prevented.
“pharmaceutically acceptable” means that the ingredients of a pharmaceutical composition are compatible with each other and not deleterious to the patient thereof.
“inhibitor of LIMK” refers to any agent that attenuates, inhibits, opposes, counteracts, or decreases the biological activity of LIMK. A LIMK antagonist may be an agent that inhibits or neutralizes LIMK biological function; an agent that prevents the binding of LIMK substrates (e.g. cofilin) to LIMK.
“selectivity ratio” refers to the ratio between a parameter corresponding to a measure of the inhibition of a kinase and the same parameter corresponding to a measure of the inhibition of LIMK. Typically, the parameter is an IC₅₀.

### DETAILED DESCRIPTION

This invention relates to compounds of Formula I

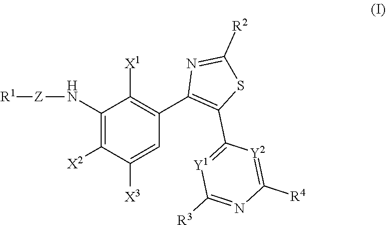

and pharmaceutically acceptable salts or solvates thereof, wherein
X¹, X² and X³ represent each independently H, halo or cyano, with the condition that at least one of X¹, X² and X³ represents halo or cyano;
Z represents a single bond, —SO₂—, —CO—CO—, —O—CR^{1′}R^{1″}—CO—, —O—CO—, oxazolyl or oxadiazolyl;
R¹ represents H, alkyl, haloalkyl, cycloalkyl, oxacycloalkyl, aryl, arylalkyl or heteroaryl, wherein alkyl, aryl, arylalkyl and heteroaryl groups are optionally substituted by one or more group selected from halo, alkyl, haloalkyl and alkoxy;
R^{1′} and R^{1″} represent each independently halo, alkyl, alkoxyalkyl, aryl or heteroaryl, wherein aryl and heteroaryl groups are optionally substituted by one or more group selected from halo, alkyl, haloalkyl and alkoxy;
R² represents H, alkyl, hydroxyalkyl, alkoxyalkyl, alkyloxycarbonylalkyl, alkylaminocarbonylalkyl or aminocarbonylalkyl;
Y¹ represents N or CH;
Y² represents N or CR⁵;
R³ represents H or NHR⁶;
R⁴ represents H, NR⁷R⁸ or R⁴ is linked with R⁵ when Y² represents CR⁵;
R⁵ represents H or R⁵ is linked with R⁴;
   wherein when R⁴ and R⁵ are linked together, —R⁴—R⁵— represents —NH—CH═CR⁹—;
R⁶ represents H or aryl, wherein the aryl group is optionally substituted by one or more halo group preferably one or more F;
R⁷ and R⁸ represent each independently H, aryl, alkyl, hydroxyalkyl, alkoxyalkyl, aminoalkyl, morpholinyl or piperazinyl;
R⁹ represents H, halo or alkyl;
provided that R³ and R⁴ are not both H;
provided that when Y¹ is CH and R³ is H, then Y² is not N;
provided that when Y¹ is N, Y² is CH, R³ is NHR⁶ and R⁴ is H, then R⁶ is not H; and provided that compound of Formula I is not N-(3-(2-(tert-butyl)-5-(1H-pyrrolo[2,3-b]pyridin-4-yl)thiazol-4-yl)-2-fluorophenyl)-2,6-difluorobenzenesulfonamide.

Advantageously, the —NH— group adjacent to —Z— moiety is deprotonated at physiological pH.

At least one of X¹, X² and X³ represents an electro-withdrawing group, such as for example halo or cyano, preferably halo, more preferably Cl or F. In one embodiment, X¹ represents halo or cyano and X² and X³ are H, preferably X¹ represent halo, especially F and X² and X³ are H. In another embodiment, X² represents halo or cyano and X¹ and X³ are H, preferably X² represent halo, especially F and X¹ and X³ are H. In another embodiment, X³ represents halo or cyano and X² and X¹ are H, preferably X³ represent halo, especially F and X² and X¹ are H.

According to one embodiment, in Formula I, R¹ represents H, alkyl, haloalkyl, cycloalkyl, oxacycloalkyl, aryl, arylalkyl or heteroaryl, wherein alkyl, haloalkyl, aryl, cycloalkyl, oxacycloalkyl, arylalkyl and heteroaryl groups are optionally substituted by one or more group selected from halo, alkyl, haloalkyl and alkoxy; preferably R¹ represents alkyl, haloalkyl, cycloalkyl or aryl, optionally substituted by one or more, preferably 1 to 5, group selected from halo and alkoxy. According to a specific embodiment, R¹ represents an alkyl group, preferably a linear or branched C3-C5-alkyl group, optionally substituted by one or more, preferably 1 to 5, alkoxy group. According to a specific embodiment, R¹ represents a haloalkyl group, preferably a linear C3-haloalkyl group. According to a specific embodiment, R¹ represents a cycloalkyl group, preferably cyclopropyl. According to another specific embodiment, R¹ represents an aryl group, optionally substituted by one or more, preferably 1 to 5, halo group, preferably R¹ represents 2,6-difluorophenyl.

According to one embodiment, in Formula I, Z represents a single bond, —SO₂—, —CO—CO—, —O—CR^{1′}R^{1″}—CO—, —O—CO—, oxazolyl or oxadiazolyl. When Z represents oxazolyl or oxadiazolyl, it corresponds to the following moieties:

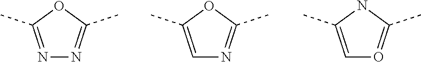

In a specific embodiment, Z represents —SO₂—, —CO—CO—, —O—CR^{1′}R^{1″}—CO— or —O—CO—, preferably Z represents —SO₂—, —CO—CO— or —O—CO—, more preferably Z represents —SO₂—. According to a preferred embodiment, Z represents-SO₂—.

According to one embodiment, in Formula I, R² represents H, alkyl, hydroxyalkyl, alkoxyalkyl, alkyloxycarbonylalkyl, alkylaminocarbonylalkyl or aminocarbonylalkyl. According to one embodiment R² represents a C2-C4 alkyl, a C2-C4 hydroxyalkyl, a alkyloxycarbonylalkyl or an alkylaminocarbonylalkyl, more preferably R² represents tert-butyl, hydroxypropyl, —CH₂—COOEt or —CH₂—CONHCH₃, even more preferably, R² represents tert-butyl.

In compounds of Formula I, the definitions of Y¹, Y², R³ and R⁴ are such that the pyridine derivative moiety linked to the thiazole ring has a formula selected from U0, U1a, U1b, U2A, U2b, U3a, U3b, U4, U5, U6, U7a, U7b, U7c, U7 d and U8:

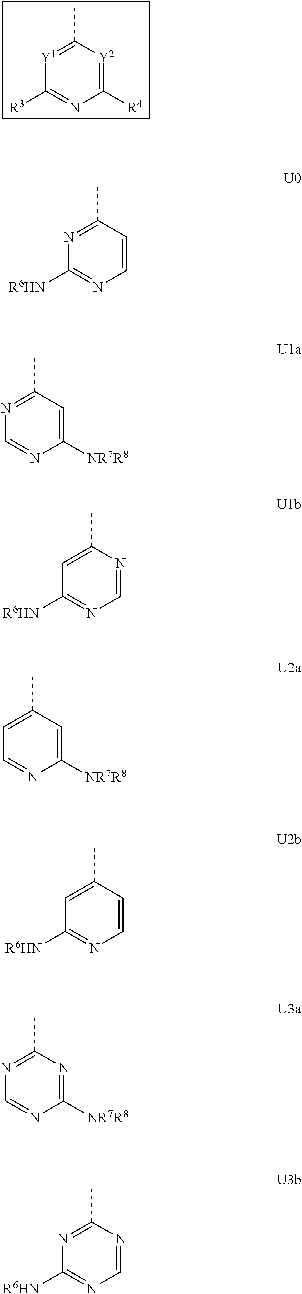

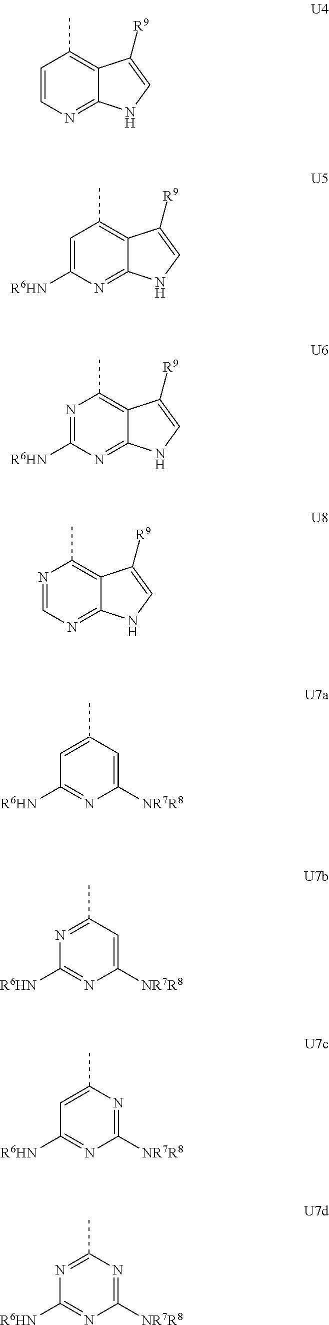

wherein R⁶, R⁷, R⁸ and R⁹ are as defined in Formula I.

All above moieties have in common a pyridine-derived core (pyridin-4yl, pyrimidin-4-yl, 1,3,5-triazin-4-yl) substituted by at least one amine group, which in some cases forms a bicyclic scaffold (pyrrolo[2,3-b]pyridin-4-yl, pyrrolo[2,3-d]pyrimidin-4-yl).

Particularly preferred pyridine derivative moieties are moiety U0, U1a, U1b, U3a, U3b, and U8, even more preferably U0 and U8.

According to one embodiment, in Formula I, R⁶ represents H, except in cases wherein Y¹ is N, Y² is CH and R⁴ is H. According to another embodiment, R⁶ represents an aryl group, wherein the aryl group is optionally substituted by one or more halo group preferably one or more F. In an embodiment, R⁶ represents a non-substituted aryl group.

According to one embodiment, in Formula I, R⁷ and R⁸ represent each independently H, aryl, alkyl or a solubilizing group such as for example hydroxyalkyl, alkoxyalkyl, aminoalkyl, morpholinyl or piperazinyl. In an embodiment, when R⁷ or R⁸ represent aryl group, then R⁷ or R⁸ represent a non-substituted aryl group.

According to one embodiment, in Formula I, R⁹ represents H. In another embodiment, in Formula I, R⁹ represents an alkyl group, such as for example methyl. In another embodiment, in Formula I, R⁹ represents halo, such as for example Cl.

In one embodiment, compounds of Formula I are of Formula Ia, Ib, Ic, Id or Ie:

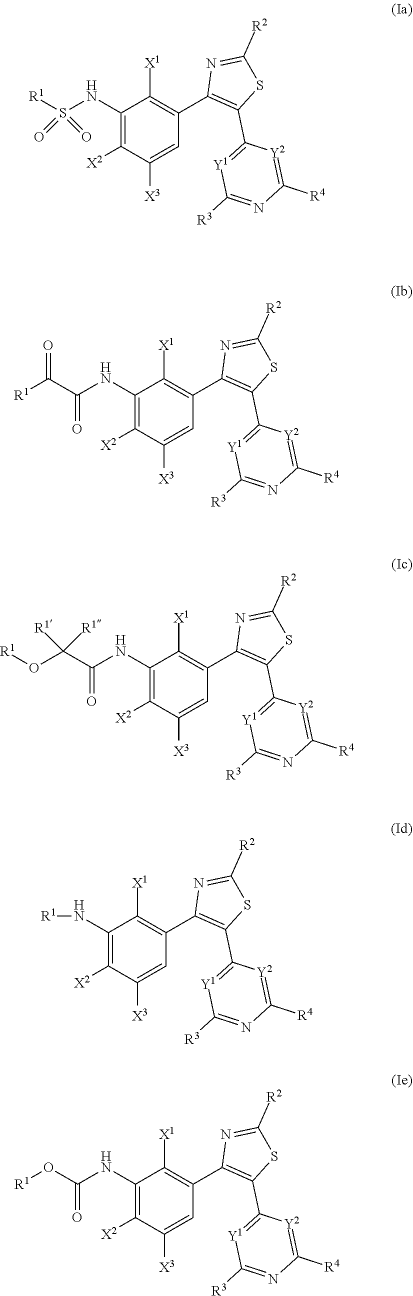

and pharmaceutically acceptable salts or solvates thereof, wherein R¹, R^{1′}, R^{1″}, R², R³, R⁴, X¹, X², X³, Y¹ and Y² are as defined above.

In one embodiment, in Formulae Ia, Ib, Ic, Id or Ie, X² and X³ are H.

In one embodiment, compounds of Formula Ia are of Formula Ia-U0, Ia-U1a, Ia-U1b, Ia-U3a, a-U3b or Ia-U8:

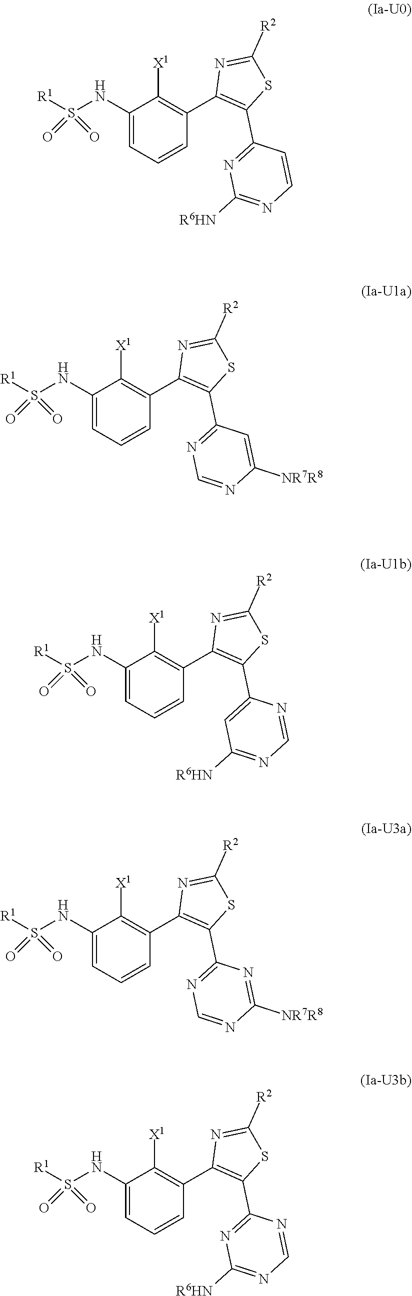

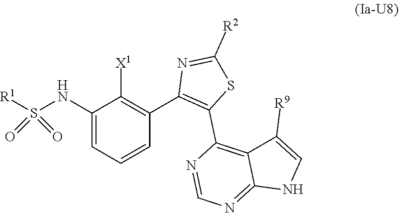

and pharmaceutically acceptable salts or solvates thereof, wherein R¹, R², R⁶, R⁷, R⁸, R⁹ and X¹ are as defined above.

In one embodiment, particularly preferred compounds of Formula Ia are of Formula Ia-U0 as defined above.

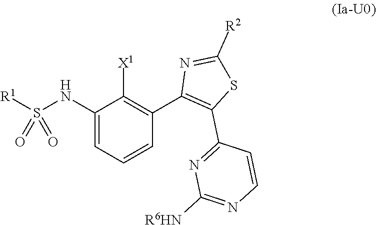

and pharmaceutically acceptable salts or solvates thereof, wherein X¹, R¹, R² and R⁶ are as defined above.

Compounds having a chemical structure close to those of the present invention are disclosed in the prior art as Raf kinase inhibitors, more specifically as B-Raf inhibitors, for example in CN103936730, WO2014/194127, WO2012/113774, WO2011/161216, WO2011/059610, WO2010/104899 and WO2009/137391.

LIM and Raf kinases are acting downstream of Receptors Tyrosine Kinase (RTK) and are phylogenically close (Manning et al., Science, 2002, 298, 1912-1934). However, it is clearly established that LIM and Raf kinases have distinct roles in signaling pathways, leading to different outcomes regarding to their respective inhibitions. Indeed, Raf kinases main target is the MEK/ERK pathway which controls proliferation, differentiation and survival through different mechanisms implying direct substrates phosphorylations but also broad transcriptional modifications via the activation of different transcription factors. In contrast, LIM kinases are the most downstream kinases in the Rho/LIMK pathway. LIM kinases mainly regulate cytoskeleton dynamics through cofilin regulation.

These differences in signaling pathways locations and in substrate specificity lead to different phenotypes upon inhibition (by small molecules, RNA interference or KO models). For example, these differences lead to different contributions for leukemogenesis which are reflected, in clinics, by different transcriptomic profiling of leukemic patients, with different patterns of Raf/LIM Kinases mRNA expressions.

Providing selective inhibitors of LIMK, especially selective over Raf kinase is interesting, especially in order to avoid off-target and side effects.

As evidenced in the experimental part, compounds of the invention are selective inhibitors of LIMK over Raf kinase, especially over B-Raf kinase.

According to one embodiment, the selectivity ratio for LIMK1 over B-Raf, calculated by dividing measured IC₅₀ B-Raf by measured IC₅₀ LIMK1, is higher than 2, preferably higher than 4, more preferably higher than 6, furthermore preferably higher than 8, furthermore preferably higher than 10. In one embodiment, compounds of Formula Ia-U0 are of Formula Ia-U0-1:

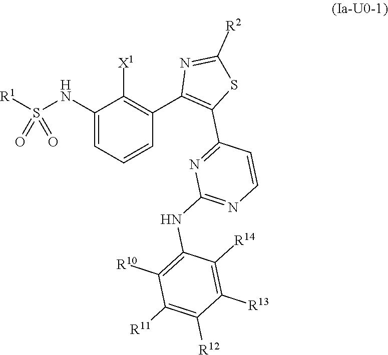

and pharmaceutically acceptable salts or solvates thereof, wherein
X¹, R¹ and R² are as defined in Formula I, and
R¹⁰, R¹¹, R¹², R¹³ and R¹⁴ represent each independently H or halo.

According to one embodiment, in Formula Ia-U0-1, R¹⁰, R¹¹, R¹², R¹³ and R¹⁴ represent all H, so that compounds of Formula Ia-U0 are of Formula Ia-U0-1′:

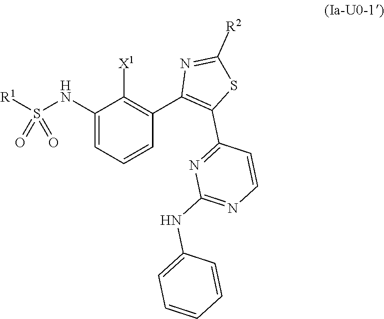

and pharmaceutically acceptable salts or solvates thereof, wherein
X¹, R¹ and R² are as defined in Formula I.

According to one embodiment, in Formula Ia-U0-1, when one or more of R¹⁰, R¹¹, R¹², R¹³ and R¹⁴ represents halo, it preferably represents a fluorine atom.

Particularly preferred compounds of Formula I are those listed in Table 1 hereafter

TABLE 1

| Cpd | | |
|---|---|---|
| n^{o} | Structure | Chemical name |
| 1 | 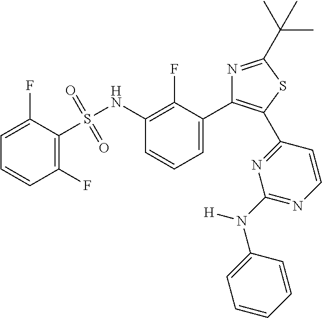 [SMILES: CC(C)(C)c1nc(-c2cccc(NS(=O)(=O)c3c(F)cccc3F)c2F)c(-c2ccnc(Nc3ccccc3)n2)s1] | N-(3-(2-(tert-butyl)-5-(2- (phenylamino)pyrimidin-4-yl)thiazol- 4-yl)-2-fluorophenyl)-2,6- difluorobenzenesulfonamide |
| 2 | 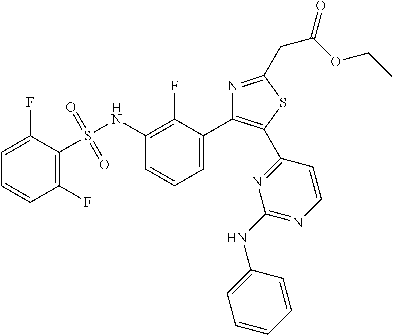 [SMILES: CCOC(=O)Cc1nc(-c2cccc(NS(=O)(=O)c3c(F)cccc3F)c2F)c(-c2ccnc(Nc3ccccc3)n2)s1] | ethyl 2-(4-(3-(2,6- difluorophenylsulfonamido)-2- fluorophenyl)-5-(2- (phenylamino)pyrimidin-4-yl)thiazol- 2-yl)acetate |
| 3 | 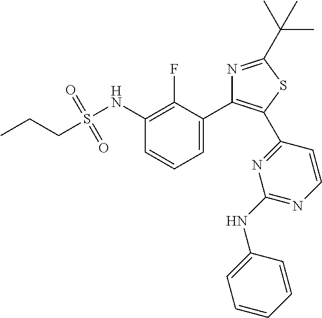 [SMILES: CCCS(=O)(=O)Nc1cccc(-c2nc(C(C)(C)C)sc2-c2ccnc(Nc3ccccc3)n2)c1F] | N-(3-(2-(tert-butyl)-5-(2- (phenylamino)pyrimidin-4-yl)thiazol- 4-yl)-2-fluorophenyl)propane-1- sulfonamide |
| 4 | 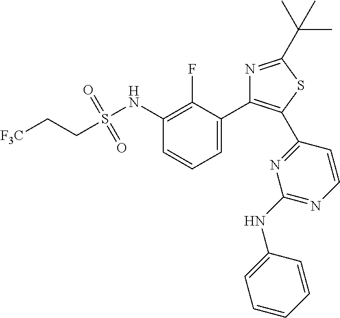 [SMILES: CC(C)(C)c1nc(-c2cccc(NS(=O)(=O)CCC(F)(F)F)c2F)c(-c2ccnc(Nc3ccccc3)n2)s1] | N-(3-(2-(tert-butyl)-5-(2- (phenylamino)pyrimidin-4-yl)thiazol- 4-yl)-2-fluorophenyl)-3,3,3- trifluoropropane-1-sulfonamide |
| 5 | 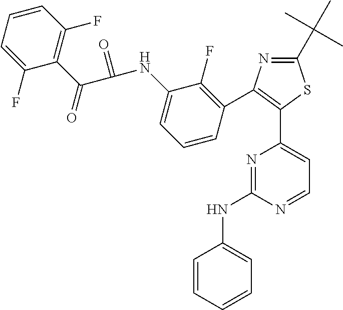 [SMILES: CC(C)(C)c1nc(-c2cccc(NC(=O)C(=O)c3c(F)cccc3F)c2F)c(-c2ccnc(Nc3ccccc3)n2)s1] | N-(3-(2-(tert-butyl)-5-(2- (phenylamino)pyrimidin-4-yl)thiazol- 4-yl)-2-fluorophenyl)-2-(2,6- difluorophenyl)-2-oxoacetamide |
| 6 | 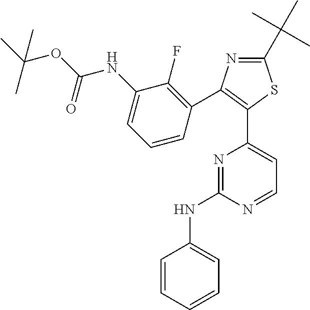 [SMILES: CC(C)(C)OC(=O)Nc1cccc(-c2nc(C(C)(C)C)sc2-c2ccnc(Nc3ccccc3)n2)c1F] | tert-butyl (3-(2-(tert-butyl)-5-(2- (phenylamino)pyrimidin-4-yl)thiazol- 4-yl)-2-fluorophenyl)carbamate |
| 7 | 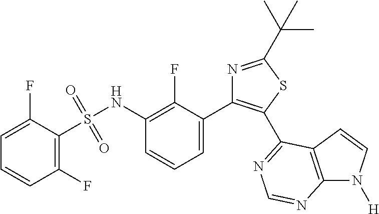 [SMILES: CC(C)(C)c1nc(-c2cccc(NS(=O)(=O)c3c(F)cccc3F)c2F)c(-c2ncnc3[nH]ccc23)s1] | N-(3-(2-(tert-butyl)-5-(7H-pyrrolo[2,3- d]pyrimidin-4-yl)thiazol-4-yl)-2- fluorophenyl)-2,6- difluorobenzenesulfonamide |
| 8 | 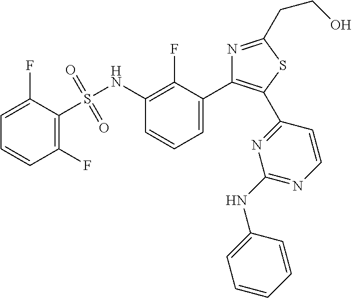 [SMILES: O=S(=O)(Nc1cccc(-c2nc(CCO)sc2-c2ccnc(Nc3ccccc3)n2)c1F)c1c(F)cccc1F] | 2,6-difluoro-N-(2-fluoro-3-(2-(2- hydroxyethyl)-5-(2- (phenylamino)pyrimidin-4-yl)thiazol- 4-yl)phenyl)benzenesulfonamide |
| 9 | 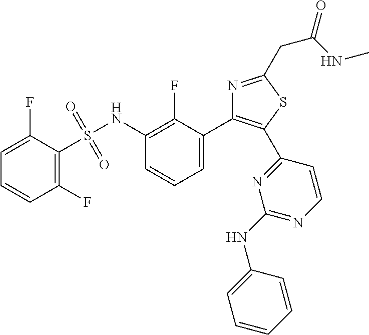 [SMILES: CNC(=O)Cc1nc(-c2cccc(NS(=O)(=O)c3c(F)cccc3F)c2F)c(-c2ccnc(Nc3ccccc3)n2)s1] | 2-(4-(3-(2,6- difluorophenylsulfonamido)-2- fluorophenyl)-5-(2- (phenylamino)pyrimidin-4-yl)thiazol- 2-yl)-N-methylacetamide |
| 10 | 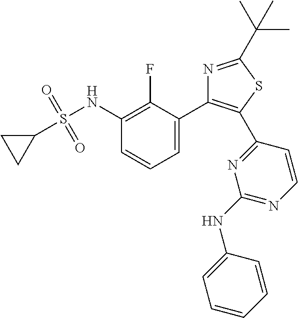 [SMILES: CC(C)(C)c1nc(-c2cccc(NS(=O)(=O)C3CC3)c2F)c(-c2ccnc(Nc3ccccc3)n2)s1] | N-(3-(2-(tert-butyl)-5-(2- (phenylamino)pyrimidin-4-yl)thiazol- 4-yl)-2- fluorophenyl)cyclopropanesulfonamide |

and pharmaceutically acceptable salts and solvates thereof.

More preferred compounds of Formula I are compounds 1, 2, 5, 8 and 9 listed in Table 1, and pharmaceutically acceptable salts and solvates thereof. Further preferred compounds of Formula I are compounds 1, 8 and 9 listed in Table 1, and pharmaceutically acceptable salts and solvates thereof.

In Table 1, the term “Cpd” means compound. The compounds of Table 1 were named using ChemBioDraw® Ultra version 12.0 (PerkinElmer).

Bonds from an asymmetric carbon in compounds of the invention are generally depicted using a solid line (—), a solid wedge ( [symbol] ), or a dotted wedge ( [symbol] ). The use of either a solid or dotted wedge to depict bonds from an asymmetric carbon atom is meant to indicate that only the stereoisomer shown is meant to be included.

The compounds of the invention include compounds of Formula I as hereinbefore defined, including salts, solvates, multi-component complexes, liquid crystals, polymorphs and crystal habits thereof, prodrugs, prodrugs and tautomers thereof and isotopically-labeled compounds of Formula I.

The compounds of the invention may be in the form of pharmaceutically acceptable salts. Pharmaceutically acceptable salts include the acid addition salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

The compounds of the invention may be prepared in salt form through the use of salt-formers. Suitable acids are preferably but not limited to those that are considered to form pharmaceutically acceptable salts (see for example: Wermuth, C. G.; Stahl, P. H. In “Handbook of Pharmaceutical Salts”, Wiley-VCH: New York, 2002). Such salts may be formed to enhance chemical purity and/or enhance storage lifetime of the attendant salt intermediate. Examples of relevant salt-formers as aforementioned include in a non-limiting sense the following acids; through any and all stereoisomeric forms where applicable: HCl, sulfuric acid, phosphoric acid, acetic acid, ethanesulfonic acid, citric acid, lactic acid, maleic acid, mandelic acid, succinic acid, phenylpropionic acid, p-toluenesulfonic acid. Preferred salt-formers include HCl.

Pharmaceutically acceptable salts of compounds of Formula I may be prepared by one or more of these methods:
by reacting the compound of Formula I with the desired acid;
by removing an acid-labile protecting group from a suitable precursor of the compound of Formula I; or
by converting one salt of the compound of Formula I to another by reaction with an appropriate acid or by means of a suitable ion exchange column.

In addition, although generally, with respect to the salts of the compounds of the invention, pharmaceutically acceptable salts are preferred, it should be noted that the invention in its broadest sense also included non-pharmaceutically acceptable salts, which may for example be used in the isolation and/or purification of the compounds of the invention. For example, salts formed with optically active acids or bases may be used to form diastereoisomeric salts that can facilitate the separation of optically active isomers of the compounds of Formula I.

Prototropic tautomer equilibrium form may exist in certain compounds of Formula I thereby engendering either or both tautomers to exist. All tautomeric forms of compounds of the invention fall, wherever applicable, within the scope of the invention regardless of which specific tautomer is drawn or named.

The term “prodrug” as used herein means the pharmacologically acceptable derivatives of compounds of Formula I, such as for example esters, whose in vivo biotransformation product generates the biologically active drug. Prodrugs are generally characterized by increased bio-availability and are readily metabolized into biologically active compounds in vivo.

The term “predrug”, as used herein, means any compound that will be modified to form a drug species, wherein the modification may take place either inside or outside of the body, and either before or after the predrug reaches the area of the body where administration of the drug is indicated.

The compounds of Formula I can be prepared by different ways with reactions known to a person skilled in the art.

The invention further provides a process of manufacturing compounds of Formula I

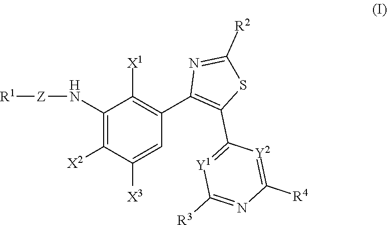

wherein R¹, R², R³, R⁴, X¹, X², X³, Y¹, Y² and Z are as defined above; comprising the following steps:
   a) reacting intermediate (A)

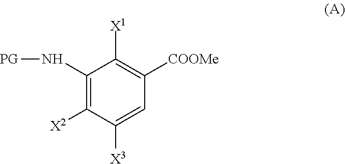

wherein PG represents an amino-protecting group;
   with intermediate (B)

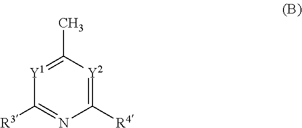

wherein R^{3′} and R^{4′} independently either represent R³ and R⁴ as defined above, or a protected precursor of respectively R³ or R⁴;
   in presence of a strong base, to afford intermediate (C)

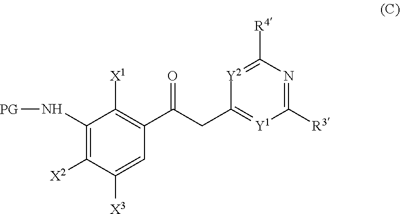

b) forming a thiazole ring by reacting intermediate (C) in presence of N-bromosuccinimide and intermediate (D)

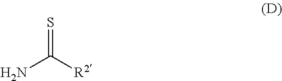

wherein R^{2′} either represents R² as defined above, or a precursor of R²;
   to afford intermediate E

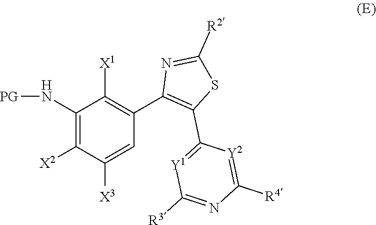

c) deprotecting intermediate (E) in conditions adapted to PG, to afford intermediate (F)

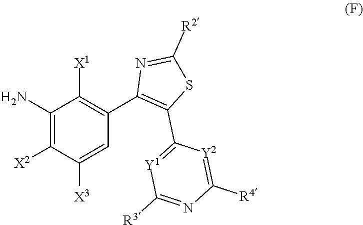

d) introducing R¹ moiety on intermediate (F) by suitable coupling reaction adapted to —Z— linker to afford compound of Formula I′

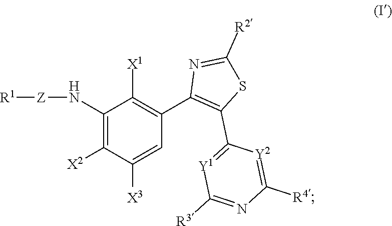

and in case wherein R^{2′}, R^{3′} and/or R^{4′} represent precursors of respectively R², R³ or R⁴, performing one or more additional intermediate steps or final steps of conversion of R^{2′} into R² and/or R^{3′} into R³ and/or of R^{4′} into R⁴.

The term “amino-protecting group” refers to a protecting group for an amine function. Suitable amino-protecting groups are known by one skilled in the art, as well as corresponding deprotection conditions. According to a preferred embodiment, the amino-protecting group is selected in the groups comprising: tert-butoxy carbonyl (Boc), arylsulphonyl, methoxymethyl, para-methoxy benzyl or benzyl. In one embodiment, the amino protecting group is Boc. In this case, deprotection may be conducted in acidic conditions.

According to one embodiment, the strong base used in step a) is lithium bis(trimethylsilyl)amide (LiHMDS).

In step d), if Z represent —SO₂—, coupling is performed in presence of intermediate (G), R¹—SO₂—Cl, preferably in presence of pyridine. The solvent of reaction is preferably tetrahydrofuran.

In step d), if Z represents —CO—CO—, coupling is performed in presence of intermediate (H), R¹—CO—COOH. In this case, coupling is preferably performed in presence of N-(3-Dimethylaminopropyl)-N′-ethylcarbodiimide hydrochloride, 2-Pyridinol 1-oxide and triethylamine. The solvent of reaction is preferably dimethylformamide.

In step d), if Z represents —O—CO—, intermediate (E) might directly correspond to a compound of Formula I, for example when PG represent a Boc group (tBu-O—CO—), being equivalent to R¹—Z— wherein R¹ is an alkyl group and Z is —O—CO—.

Reaction schemes as described in the example section are illustrative only and should not be construed as limiting the invention in any way.

Methods of Inhibiting LIMK

The invention also relates to a method of inhibiting LIMK activity (e.g. LIMK 1 activity and/or LIMK 2 activity), in vitro or in vivo, comprising contacting LIMK (e.g. LIMK 1 and/or LIMK 2) with an effective amount of a compound of Formula I according to the invention.

According to one embodiment, the invention relates to a method of inhibiting LIMK activity (e.g. LIMK 1 activity and/or LIMK 2 activity) in a cell, in vitro or in vivo, comprising contacting the cell with an effective amount of a compound of Formula I according to the invention.

Suitable assays for determining LIMK activity inhibition are described herein and/or are known in the art.

According to a further feature of the present invention there is provided the use of a compound of Formula I or a pharmaceutically acceptable salt or solvate thereof for the manufacture of a medicament for modulating (e.g., inhibiting) LIMK activity in a patient, in need of such treatment, which comprises administering to said patient an effective amount of a compound of the invention, or a pharmaceutically acceptable salt or solvate thereof.

Preferably, the patient is a warm-blooded animal, more preferably a human.

Methods of Inhibiting Cell Proliferation

The compounds of Formula I described herein (a) regulate (e.g., inhibit) cell proliferation; (b) inhibit cell cycle progression; (c) promote apoptosis; or (d) a combination of one or more of these.

According to one embodiment, the invention relates to a method of regulating (e.g., inhibiting) cell proliferation, inhibiting cell cycle progression, promoting apoptosis, or a combination of one or more these, in vitro or in vivo, comprising contacting a cell with an effective amount of a compound of Formula I according to the invention.

Suitable assays for determining whether or not a compound inhibits cell proliferation are described herein and/or are known in the art.

Use in the Treatment and/or Prevention of LIMK-Related Diseases

The present invention also relates to a medicament comprising at least one compound of Formula I or a pharmaceutically acceptable salt or solvate thereof as active ingredient.

The invention further provides the use of a compound of Formula I or a pharmaceutically acceptable salt or solvate thereof for the manufacture of a medicament for treating and/or preventing LIMK-related diseases.

The compounds of the invention are therefore useful as medicaments, in particular in the prevention and/or treatment of LIMK-related diseases. According to one embodiment, the invention thus relates to a compound of Formula I or a pharmaceutically acceptable salt or solvate thereof for use in the prevention and/or treatment of LIMK-related diseases.

The invention also provides for a method for delaying in patient the onset of a LIMK-related disease.

Preferably, the patient is a warm-blooded animal, more preferably a human.

Another aspect of the present invention pertains to a method of treatment of a LIMK-related disease, comprising administering to a patient in need of treatment a therapeutically effective amount of a compound of Formula I, as described herein.

LIMK-Related Diseases

The term “LIMK-related disease” refers to any disease in which LIMK is known to play a role. It also means any disease which is alleviated by treatment with a LIMK inhibitor.

In one embodiment the LIMK-related diseases is selected from:
proliferative conditions (see details below);
neurodegenerative or neurodevelopmental disorders, such as for example Alzheimer's disease, Parkinson's disease, Williams syndrome;
cardiovascular and vascular diseases, such as for example hypertension, pulmonary hypertension, pulmonary vasoconstriction, angina, cerebral vasospasm, ischemia following subarachnoid hemorrhage, intracranial aneurism, atherosclerosis;
eye diseases, such as for example: glaucoma, degenerative retinal diseases such as macular degeneration, vision loss due to diabetic macular edema, vision loss due to macular edema secondary to retinal vein occlusion proliferative vitreoretinopathy; inflammatory eye diseases such as anterior uveitis, panuveitis, intermediate uveitis and posterior uveitis, glaucoma filtration surgery failure, dry eye, allergic conjunctivitis, posterior capsule opacification, cataract formation, abnormalities of corneal wound healing, ocular pain and ocular hypertension;
airway diseases, such as for example chronic obstructive pulmonary disease (COPD), fibrosis, pulmonary fibrosis, emphysema, chronic bronchitis, asthma, pneumonia, cystic fibrosis, bronchitis and rhinitis and respiratory distress syndrome;
inflammatory diseases, such as for example Crohn's disease, ulcerative colitis, contact dermatitis, atopic dermatitis, rheumatoid arthritis, juvenile rheumatoid arthritis, ankylosing spondylitis, psoriatic arthritis, inflammatory bowel disease;
skin diseases, such as for example scarring, hyperkeratosis, parakeratosis, hypergranulosis, acanthosis, dyskeratosis, spongiosis and ulceration;
intestinal diseases, such as for example inflammatory bowel disease (IBD), colitis, gastroenteritis, ileus, ileitis, appendicitis and Crohn's disease;
kidney diseases, such as for example renal fibrosis or renal dysfunction;
bone diseases, such as for example osteoporosis and osteoarthritis;
viral diseases, such as for example a retroviral disease, more in particular human immunodeficiency virus (HIV);
drug addiction;
neurofibromatosis.

The term “proliferative condition” refers to an unwanted or uncontrolled cellular proliferation of excessive or abnormal cells which is undesired, such as, neoplastic or hyperplastic growth.

In one embodiment, the proliferative condition is characterized by benign, pre-malignant, or malignant cellular proliferation, including but not limited to, neoplasms, hyperplasias, and tumors (e.g., histocytoma, glioma, astrocytoma, osteoma), cancers (see below), psoriasis, bone diseases, fibroproliferative disorders (e.g., of connective tissues), pulmonary fibrosis, atherosclerosis, smooth muscle cell proliferation in the blood vessels, such as stenosis or restenosis following angioplasty.

In one embodiment, the LIMK-related disease is a cancer characterised by, or further characterised by, cancer cells which overexpress LIM kinase (LIMK) (e.g., LIMK1 and/or LIMK2). In another embodiment, the LIMK-related disease is a cancer characterised by, or further characterised by, a progression linked to LIM kinase (LIMK) (e.g., LIMK1 and/or LIMK2) but without LIMK overexpression, such as for example in some leukemias.

In one embodiment, the treatment is treatment of lung cancer, small cell lung cancer, non-small cell lung cancer, gastrointestinal cancer, stomach cancer, bowel cancer, colon cancer, rectal cancer, colorectal cancer, thyroid cancer, breast cancer, ovarian cancer, endometrial cancer, uterus cancer, ovary cancer, prostate cancer, testicular cancer, liver cancer, kidney cancer, pancreas cancer, renal cell carcinoma, bladder cancer, pancreatic cancer, brain cancer, nerve cancer, glioma, sarcoma, osteosarcoma, bone cancer, nasopharyngeal cancer (e.g., head cancer, neck cancer), skin cancer, squamous cancer, Kaposi's sarcoma, melanoma, malignant melanoma, lymphoma, or leukemia.

In one embodiment, the cancer is selected from:
a carcinoma, for example a carcinoma of the bladder, breast, colon (e.g., colorectal carcinomas such as colon adenocarcinoma and colon adenoma), bowel, rectum, kidney, epidermal, liver, lung (e.g., adenocarcinoma, small cell lung cancer and non-small cell lung carcinomas), esophagus, gall bladder, ovary, uterus, endometrium, pancreas (e.g., exocrine pancreatic carcinoma), stomach, cervix, thyroid, prostate, testicle, skin (e.g., squamous cell carcinoma), brain, nerve, bone;
a hematopoietic tumor of lymphoid lineage, for example leukemia, acute lymphocytic leukemia, B-cell lymphoma, T-cell lymphoma, Hodgkin's lymphoma, non-Hodgkin's lymphoma, hairy cell lymphoma, or Burkett's lymphoma;
a hematopoietic tumor of myeloid lineage, for example acute myeloid leukemia (including acute promyelocytic leukemia), chronic myeloid leukemia or myelodysplasia syndrome;
a tumor of mesenchymal origin, for example fibrosarcoma or rhabdomyosarcoma;
a tumor of the central or peripheral nervous system, for example astrocytoma, neuroblastoma, glioma or schwannoma;
melanoma; seminoma; teratocarcinoma; osteosarcoma; xeroderma pigmentosum;
keratoacanthoma; thyroid follicular cancer; or Kaposi's sarcoma.

In one embodiment, the LIMK-related disease is cancer metastasis, especially metastatic breast cancer.

In one embodiment, the LIMK-related disease is acute myeloid leukemia (AML). According to a specific embodiment, treated patients are diagnosed as suffering from an acute myeloid leukemia with FLT3 mutations.

In one embodiment, the LIMK-related disease is sarcoma.

Combination Therapy

According to one embodiment, the compounds of the invention, their pharmaceutical acceptable salts or solvates may be administered as part of a combination therapy. Thus, are included within the scope of the present invention embodiments comprising coadministration of, and compositions and medicaments which contain, in addition to a compound of the present invention or a pharmaceutically acceptable salt or solvate thereof as active ingredient, additional therapeutic agents and/or active ingredients. Such multiple drug regimens, often referred to as “combination therapy”, may be used in the treatment and/or prevention of any of the diseases or conditions mediated by or associated with LIMK modulation. The use of such combinations of therapeutic agents is especially pertinent with respect to the treatment of the above-mentioned disorders within a patient in need of treatment or one at risk of becoming such a patient.

In addition to the requirement of therapeutic efficacy, which may necessitate the use of active agents in addition to the LIMK inhibitor compounds of Formula I or pharmaceutical acceptable salts and solvates thereof, there may be additional rationales which compel or highly recommend the use of combinations of drugs involving active ingredients which represent adjunct therapy, i.e., which complement and supplement the function performed by the LIMK inhibitor compounds of the present invention. Suitable supplementary therapeutic agents used for the purpose of auxiliary treatment include drugs which, instead of directly treating or preventing a disease or condition mediated by or associated with LIMK modulation, treat diseases or conditions which directly result from or indirectly accompany the basic or underlying LIMK modulated disease or condition.

According to a further feature of the present invention, the compound of Formula I, a pharmaceutically acceptable salt or solvate thereof may be used in combination therapy with for example anthracycline compounds (particularly but not exclusively daunorubicin
including in its liposomal formulation-, doxorubicin, idarubicin, mitoxanthrone), ATRA, arsenic trioxide, alkylating drugs (particularly but not exclusively melphalan, cyclophosphamide, amsacrin, busulfan, treosulfan), cytarabine and derivatives thereof (particularly but not exclusively decitabine, fludarabine), kinases inhibitors (particularly but not exclusively FLT3 and/or c-Kit inhibitors), Bcl-2 inhibitors, immune checkpoint inhibitors, vinca alkaloids (particularly but not exclusively vincristine and vinblastine), glucocorticoids (particularly but not exclusively prednisolone), anti-folate drugs (particularly but not exclusively methotrexate), etoposide, 6-thioguanine, 6-mercaptopurine, G-CSF, fligrastim or total body irradiation, to improve their efficacy and to minimize secondary effects associated thereto.

In the above-described embodiment combinations of the present invention, the compound of Formula I, a pharmaceutically acceptable salt or solvate thereof and other therapeutic active agents may be administered in terms of dosage forms either separately or in conjunction with each other, and in terms of their time of administration, either serially or simultaneously. Thus, the administration of one component agent may be prior to, concurrent with, or subsequent to the administration of the other component agent(s).

According to a further embodiment, the compounds of the invention, their pharmaceutical acceptable salts or solvates thereof, may be used in combination with irradiation treatments and/or surgical treatments. By “irradiation treatment” it is especially referred to radiotherapy and total body irradiation. Such combinations may be used in the treatment and/or prevention of any of the diseases or conditions mediated by or associated with LIMK modulation. The use of such combinations is especially relevant with respect to the treatment of the above-mentioned disorders within a patient in need of treatment or one at risk of becoming such a patient. In such combinations, the compound of the invention, a pharmaceutically acceptable salt or solvate thereof, may be administered either prior to, concurrent with, or subsequent to the irradiation treatment and/or the surgical treatment.

Pharmaceutical Composition

The invention also provides pharmaceutical compositions comprising a compound of Formula I or a pharmaceutically acceptable salt or solvate thereof and at least one pharmaceutically acceptable carrier, diluent, excipient and/or adjuvant. As indicated above, the invention also covers pharmaceutical compositions which contain, in addition to a compound of the present invention, a pharmaceutically acceptable salt or solvate thereof as active ingredient, additional therapeutic agents and/or active ingredients.

Generally, for pharmaceutical use, the compounds of the invention may be formulated as a pharmaceutical preparation comprising at least one compound of the invention and at least one pharmaceutically acceptable carrier, diluent, excipient and/or adjuvant, and optionally one or more further pharmaceutically active compounds.

By means of non-limiting examples, such a formulation may be in a form suitable for oral administration, for parenteral administration (such as by intravenous, intramuscular or subcutaneous injection or intravenous infusion), for topical administration (including ocular), for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is made to the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, cremes, lotions, soft and hard gelatin capsules, suppositories, drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other substances that are commonly used in pharmaceutical formulations, such as lubricating agents, wetting agents, emulsifying and suspending agents, dispersing agents, desintegrants, bulking agents, fillers, preserving agents, sweetening agents, flavoring agents, flow regulators, release agents, etc. . . . . The compositions may also be formulated so as to provide rapid, sustained or delayed release of the active compound(s) contained therein.

The pharmaceutical preparations of the invention are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 0.1 and 10 000 mg of at least one compound of the invention.

Usually, depending on the condition to be prevented or treated and the route of administration, the active compound of the invention will usually be administered between 0.001 and 150 mg per kilogram body weight of the patient per day, which may be administered as a single daily dose, divided over one or more daily doses, or essentially continuously, e.g. using a drip infusion.

According to one embodiment, the active compound of the invention will be administered as a single daily dose, divided over one, two or more daily doses, or essentially continuously, e.g. using a drip infusion.

The present invention is further illustrated by the following examples. These examples are intended to be representative of specific embodiments of the invention and are not intended as limiting the scope of the invention.

### EXAMPLES

### Abbreviations

ATP: adenosine triphosphate;
BSA: bovine serum albumin;
DMSO: dimethyl sulfoxide;
EDTA: ethylenediaminetetraacetic acid;
FBS: fetal bovine serum;
GST: glutathione S-transferase;
MOPS: 3-(N-morpholino)propanesulfonic acid;
NP-40: 4-nonylphenyl poly(ethylene glycol);
PVDF membrane: polyvinylidene difluoride membrane;
RIPA: radioimmunoprecipitation assay;
RPMI: Roswell Park Memorial Institute medium;
RT: room temperature;
SDS-PAGE: sodium dodecyl sulfate polyacrylamide gel electrophoresis;
TBS: Tris Buffered Saline.

### I. Chemistry Examples

### I.1. Synthesis of Compound 1

### Methyl 3-amino-2-fluorobenzoate

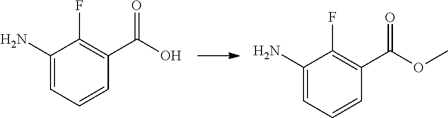

A solution of 3-amino-2-fluorobenzoic acid (1.0 g, 6.45 mmol) in MeOH (60 mL) was treated with concentrated H₂SO₄ (0.5 mL) and heated to reflux for 3 h. Another portion of concentrated H₂SO₄ (0.3 mL) was added and the medium was refluxed for 2 h. The reaction was then stirred at 50° C. over the WE. Solid sodium bicarbonate was carefully added and the methanol was evaporated under reduced pressure. The residue was partitioned between EtOAc and water. The aqueous layer was extracted with EtOAc. Combined organics were washed with brine, dried over sodium sulphate, filtered, the filtrate was concentrated under vacuum to afford the title compound (1.2 g, quantitative).

### Methyl 3-((2,6-difluorophenyl)sulfonamido)-2-fluorobenzoate

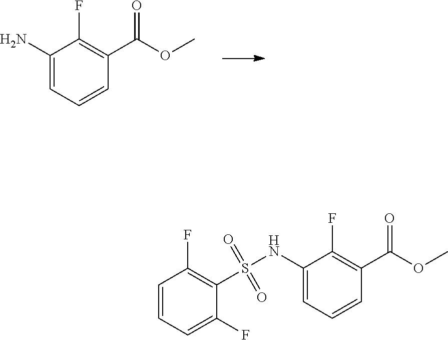 [SMILES: COC(=O)c1cccc(N)c1F ; COC(=O)c1cccc(NS(=O)(=O)c2c(F)cccc2F)c1F]

A solution of Methyl 3-amino-2-fluorobenzoate (2.0 g, 11.82 mmol) in DCM (40 mL) was treated at 0° C. with pyridine (1.91 mL, 23.65 mmol) and with 2,6-difluorobenzenesulfonyl chloride (2.40 mL, 17.17 mmol). The reaction mixture was stirred at room temperature overnight. The medium was poured onto water, and aqueous HCl (1M) was added. The aqueous layer was extracted with DCM. Combined organics were washed with saturated aqueous sodium bicarbonate, dried over sodium sulphate, filtered and the filtrate was concentrated under reduced pressure. Purification by flash chromatography on silica (DCM/cHex 8/2 to 10/0) afforded the title compound (2.55 g, 62%). LC/MS (ES⁻): 344.2 (M−1).

### N-(3-(2-(2-chloropyrimidin-4-yl)acetyl)-2-fluorophenyl)-2,6-difluorobenzenesulfonamide

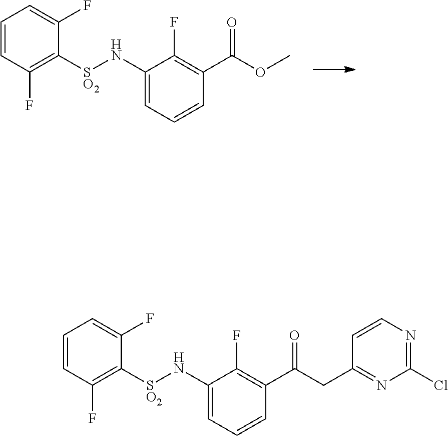 [SMILES: COC(=O)c1cccc(NS(=O)(=O)c2c(F)cccc2F)c1F ; O=C(Cc1ccnc(Cl)n1)c1cccc(NS(=O)(=O)c2c(F)cccc2F)c1F]

A solution of Lithium bis(trimethylsilyl)amide (1M in THF, 4.05 mL, 4.05 mmol) was added to a cooled (0° C.) solution of Methyl 3-((2,6-difluorophenyl)sulfonamido)-2-fluorobenzoate (400 mg, 1.16 mmol) in THF (3 mL). After 10 min of stirring, a solution of 2-chloro-4-methylpyrimidine (178 mg, 1.39 mmol) in THF (2 mL) was slowly added and the reaction mixture was allowed to warm to room temperature for 2 h. Aqueous saturated ammonium chloride was added to the medium. The aqueous layer was extracted with EtOAc (2 times). Combined organics were washed with brine, dried over sodium sulphate, filtered and the filtrate was concentrated under reduced pressure. Trituration of the brown solid in DCM/cHex afforded title compound (420 mg, 82%) as a mixture of ketone and enol. LC/MS (ES⁺): 442.0-444.0 (M+1).

### N-(3-(2-(tert-butyl)-5-(2-chloropyrimidin-4-yl)thiazol-4-yl)-2-fluorophenyl)-2,6-difluorobenzenesulfonamide

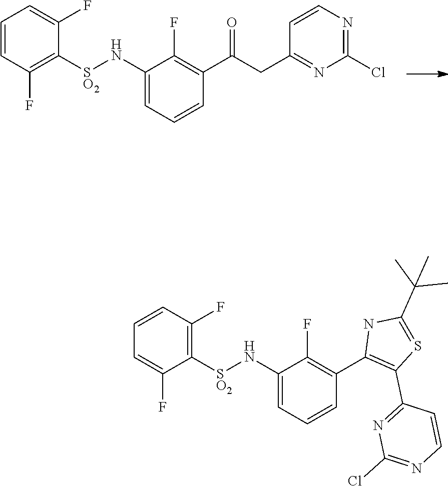

N-Bromosuccinimide (80 mg, 0.45 mmol) was added to a solution of N-(3-(2-(2-chloropyrimidin-4-yl)acetyl)-2-fluorophenyl)-2,6-difluorobenzenesulfonamide (200.0 mg, 0.45 mmol) in dimethylacetamide (2 mL). The reaction mixture was stirred at room temperature for 1 h then 2,2,2-Trimethylthioacetamide (58 mg, 0.49 mmol) was added. After stirring at room temperature for 1 h, the medium was stirred at 60° C. Once the reaction was complete, the medium was partitioned between water and EtOAC. The aqueous layer was extracted with EtOAc. Combined organics were washed with water and brine, dried over sodium sulphate, filtered and concentrated under vacuum. Purification by column chromatography on silica gel (cHex/EtOAc, 1/0 to 0/1) afforded the title compound (135 mg, 55%). LC/MS (ES⁺): 539.2-541.2 (M+1).

### N-(3-(2-(tert-butyl)-5-(2-(phenylamino)pyrimidin-4-yl)thiazol-4-yl)-2-fluorophenyl)-2,6-difluorobenzenesulfonamide (Compound 1)

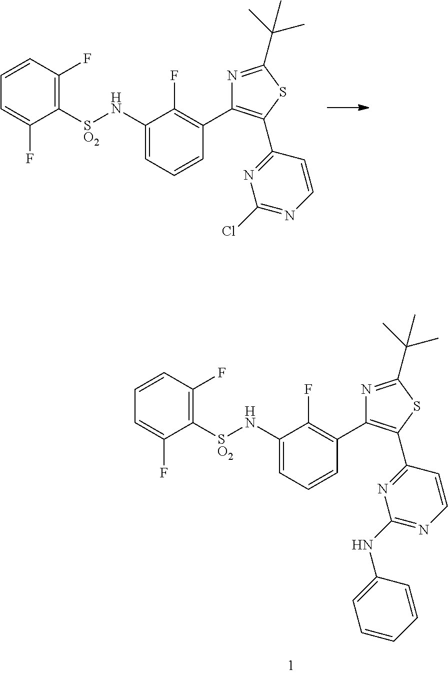 [SMILES: CC(C)(C)c1nc(-c2cccc(NS(=O)(=O)c3c(F)cccc3F)c2F)c(-c2ccnc(Cl)n2)s1 ; CC(C)(C)c1nc(-c2cccc(NS(=O)(=O)c3c(F)cccc3F)c2F)c(-c2ccnc(Nc3ccccc3)n2)s1]

A suspension of N-(3-(2-(tert-butyl)-5-(2-chloropyrimidin-4-yl)thiazol-4-yl)-2-fluorophenyl)-2,6-difluorobenzenesulfonamide (50.0 mg, 0.092 mmol) and aniline (9.0 mg, 0.097 mmol) in iPrOH (1 mL), in presence of catalytic concentrated HCl, was stirred at 100° C. for 1 h, then at 80° C. overnight. The mixture was concentrated under reduced pressure. The residue was partitioned between EtOAc and saturated aqueous sodium bicarbonate. The organic layer was washed with brine, dried over sodium sulphate, filtered and the filtrate was concentrated under vacuum. Purification by flash chromatography on silica gel (cHex-EtOAc, 1/0 to 0/1) afforded the title compound 1 (40 mg, 72%). ¹H NMR (CDCl₃): 8.09 (d, 1H, J=5.2 Hz); 7.84 (bs, 1H); 7.72 (m, 1H); 7.48 (d, 2H, J=7.7 Hz); 7.44-7.36 (m, 3H); 7.29 (t, 2H, J=7.5 Hz); 7.24 (t, 1H, J=7.3 Hz); 7.02 (t, 1H, J=7.7 Hz); 6.92 (t, 2H, J=8.7 Hz); 6.28 (d, 1H, J=5.2 Hz); 1.48 (s, 9H). LC/MS (ES⁺): 596.2 (M+1).

### I.2. Synthesis of Compound 2

### Ethyl 2-(5-(2-chloropyrimidin-4-yl)-4-(3-((2,6-difluorophenyl)sulfonamido)-2-fluorophenyl)thiazol-2-yl)acetate

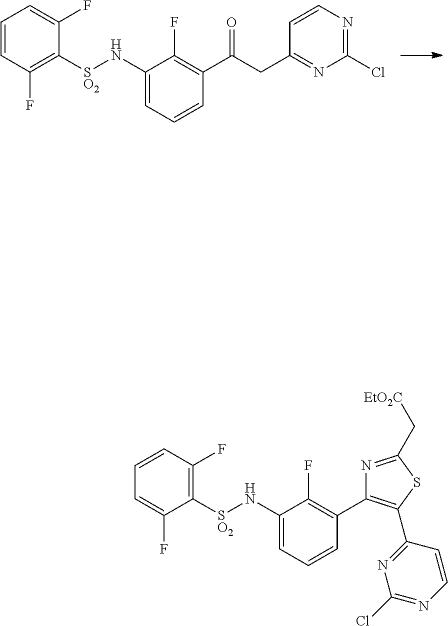 [SMILES: O=C(Cc1ccnc(Cl)n1)c1cccc(NS(=O)(=O)c2c(F)cccc2F)c1F ; CCOC(=O)Cc1nc(-c2cccc(NS(=O)(=O)c3c(F)cccc3F)c2F)c(-c2ccnc(Cl)n2)s1]

N-Bromosuccinimide (221 mg, 1.24 mmol) was added to a solution of N-(3-(2-(2-chloropyrimidin-4-yl)acetyl)-2-fluorophenyl)-2,6-difluorobenzenesulfonamide obtained as described above (500 mg, 1.13 mmol) in dimethylacetamide (4.5 mL). The reaction mixture was stirred at room temperature for 1 h then ethyl 3-amino-3-thioxopropanoate (200 mg, 1.36 mmol) was added. After stirring at room temperature for 1 h, the medium was partitioned between water and EtOAc. The organic layer was washed with water, brine, dried over sodium sulphate, filtered and the filtrate was concentrated under vacuum. Purification by column chromatography on silica gel (DCM/EtOAc, 95/5 to 0/1) afforded the title compound (160 mg, 25%). LC/MS (ES⁺): 569.5-571.5 (M+1).

### Ethyl 2-(4-(3-((2,6-difluorophenyl)sulfonamido)-2-fluorophenyl)-5-(2-(phenylamino)pyrimidin-4-yl)thiazol-2-yl)acetate (Compound 2)

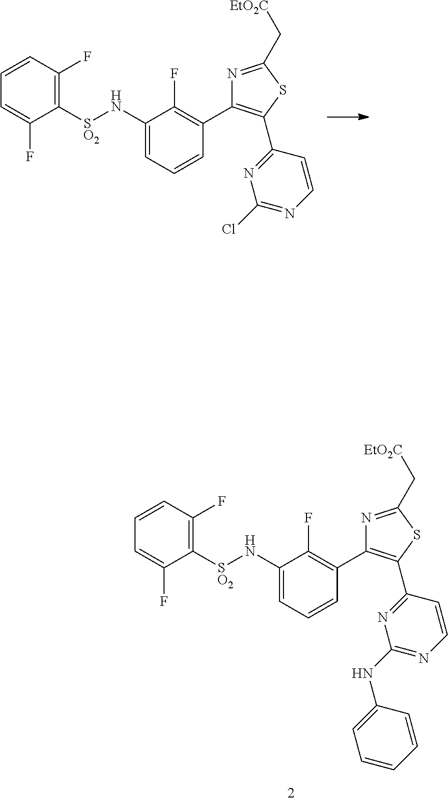 [SMILES: CCOC(=O)Cc1nc(-c2cccc(NS(=O)(=O)c3c(F)cccc3F)c2F)c(-c2ccnc(Cl)n2)s1 ; CCOC(=O)Cc1nc(-c2cccc(NS(=O)(=O)c3c(F)cccc3F)c2F)c(-c2ccnc(Nc3ccccc3)n2)s1]

A suspension of Ethyl 2-(5-(2-chloropyrimidin-4-yl)-4-(3-((2,6-difluorophenyl)sulfonamido)-2-fluorophenyl)thiazol-2-yl)acetate (200.0 mg, 0.35 mmol) and aniline (58 μL, 0.63 mmol) in iPrOH (1.5 mL), in presence of catalytic concentrated HCl, was stirred at 120° C. for 30 min, under μwaves irradiation. After cooling at 0° C., the supernatant was removed. Sonication and trituration of the remaining gum in iPrOH afforded the title compound 2 (196 mg, 89%). LC/MS (ES⁺): 626.5 (M+1).

### I.3. Synthesis of Compound 3

### 4-(4-(3-amino-2-fluorophenyl)-2-(tert-butyl)thiazol-5-yl)-N-phenylpyrimidin-2-amine

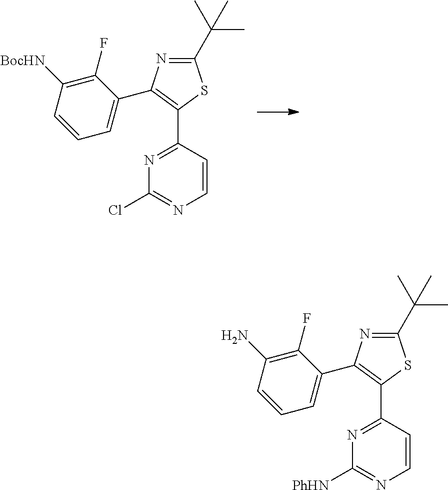

A suspension of tert-butyl (3-(2-(tert-butyl)-5-(2-chloropyrimidin-4-yl)thiazol-4-yl)-2-fluorophenyl)carbamate (cf synthesis of compound 6) (170 mg, 0.36 mmol) and aniline (51 mg, 0.55 mmol) in iPrOH (3.7 mL), in presence of concentrated HCl (34 μL), was stirred at 120° C. for 2 h, under μwaves irradiation. The mixture was concentrated under vacuum and the residue was partitioned between EtOAc and saturated aqueous sodium bicarbonate. The organic layer was washed with brine, dried over sodium sulphate, filtered and concentrated under vacuum. Purification of the residue by flash chromatography on silica gel (cHex/EtOAc 1/0 to 0/1) afforded the title compound (102 mg, 66%). LC/MS (ES⁺): 420.4 (M+1).

### N-(3-(2-(tert-butyl)-5-(2-(phenylamino)pyrimidin-4-yl)thiazol-4-yl)-2-fluorophenyl)propane-1-sulfonamide (Compound 3)

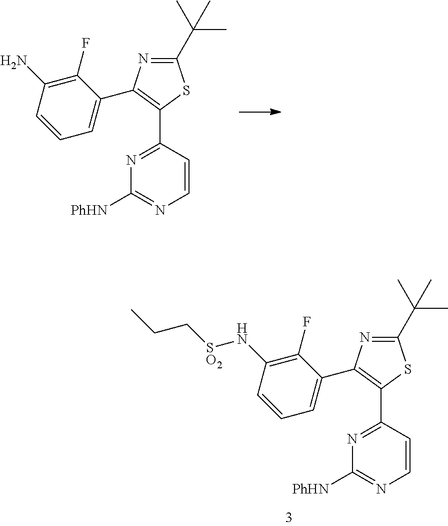

To a cooled (0° C.) solution of 4-(4-(3-amino-2-fluorophenyl)-2-(tert-butyl)thiazol-5-yl)-N-phenylpyrimidin-2-amine (47 mg, 0.11 mmol) and pyridine (10 μL, 0.12 mmol) in THF (1.1 mL) was added propyl sulfonyl chloride (14 μL, 0.12 mmol). After 1 h of heating at 50° C., the mixture was partitioned between EtOAc and aqueous HCl (0.1N). The organic layer was washed with aqueous HCl (0.1N), with brine, dried over sodium sulphate, filtered and dried under vacuum. Purification of the residue by preparative TLC (cHex-EtOAc 7/3) afforded the title compound 3 (22 mg, 37%). LC/MS (ES⁺): 526.4 (M+1).

### I.4. Synthesis of Compound 4

### N-(3-(2-(tert-butyl)-5-(2-(phenylamino)pyrimidin-4-yl)thiazol-4-yl)-2-fluorophenyl)-3,3,3-trifluoropropane-1-sulfonamide (Compound 4)

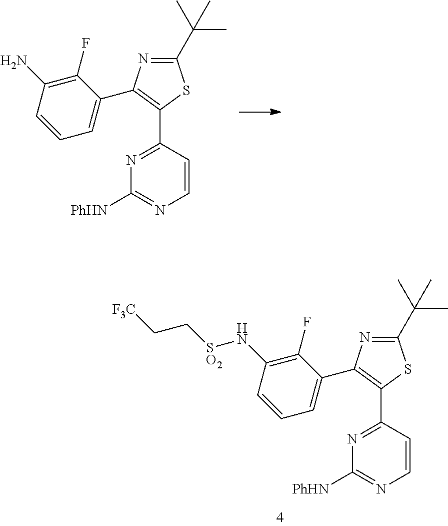

To a cooled (0° C.) solution of 4-(4-(3-amino-2-fluorophenyl)-2-(tert-butyl)thiazol-5-yl)-N-phenylpyrimidin-2-amine (30 mg, 71 μmol) and pyridine (13 μL, 157 μmol) in THF (0.7 mL) was added 3,3,3-trifluoropropane sulfonyl chloride (20 μL, 157 μmol). After heating at 50° C. overnight, the mixture was partitioned between EtOAc and aqueous HCl (0.1N). The organic layer was washed with aqueous HCl (0.1N), with brine, dried over sodium sulphate, filtered and dried under vacuum. Purification of the residue by preparative TLC (cHex-EtOAc 7/3) afforded the title compound 4 (12 mg, 31%). LC/MS (ES⁺): 580.5 (M+1).

### I.5. Synthesis of Compound 5

### N-(3-(2-(tert-butyl)-5-(2-(phenylamino)pyrimidin-4-yl)thiazol-4-yl)-2-fluorophenyl)-2-(2,6-difluorophenyl)-2-oxoacetamide (Compound 5)

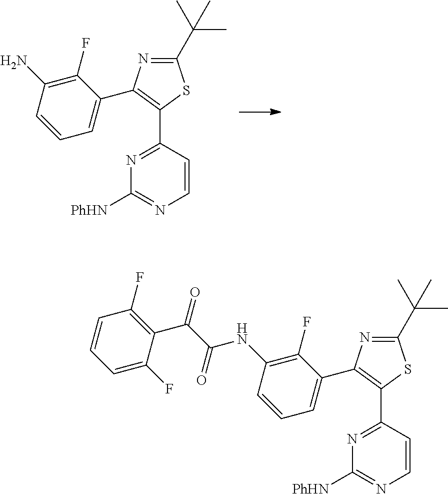

To a mixture of 4-(4-(3-amino-2-fluorophenyl)-2-(tert-butyl)thiazol-5-yl)-N-phenylpyrimidin-2-amine (20 mg, 47 μmol) and 2-(2,6-difluorophenyl)-2-oxoacetic acid (28 mg, 150 μmol) in DMF (1 mL) were added N-(3-Dimethylaminopropyl)-N′-ethylcarbodiimide hydrochloride (22 mg, 110 μmol), 2-Pyridinol 1-oxide (0.5 mg, 4 μmol) and triethylamine (20 μL, 140 μmol). After 1 h of stirring at room temperature, the mixture was partitioned between EtOAc and saturated aqueous sodium bicarbonate. The organic layer was washed with saturated aqueous sodium bicarbonate, aqueous HCl (0.1N), brine, dried over sodium sulphate, filtered and the filtrate was concentrated under vacuum. Purification of the residue by preparative TLC (cHex-EtOAc 75/25) afforded the title compound (6.4 mg, 22%). LC/MS (ES+): 588.4 (M+1).

### I.6. Synthesis of Compound 6

### Methyl 3-((tert-butoxycarbonyl)amino)-2-fluorobenzoate

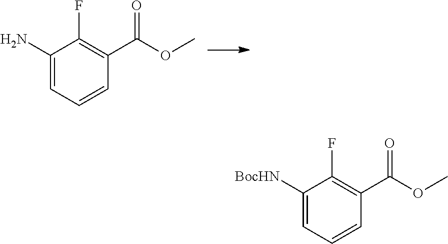 [SMILES: COC(=O)c1cccc(N)c1F ; COC(=O)c1cccc(NC(=O)OC(C)(C)C)c1F]

To a cooled solution (0° C.) of Methyl 3-amino-2-fluorobenzoate (600 mg, 3.55 mmol) in THF (177 mL) was added triphosgene (526 mg, 1.77 mmol). The reaction mixture was refluxed for 3 hours and concentrated under reduced pressure. The residue was dissolved in THF/tert-butyl alcohol (25 mL/155 mL). The resulting solution was treated with triethylamine (494 μL, 3.55 mmol) and heated at 50° C. for 2 hours. After concentration under vacuum, purification of the residue by flash chromatography on silica gel (cHex-EtOAc 1/0 to 8/2) afforded the title compound (790 mg, 81%).

### tert-butyl (3-(2-(2-chloropyrimidin-4-yl)acetyl)-2-fluorophenyl)carbamate

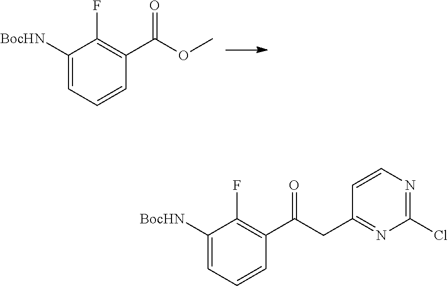 [SMILES: COC(=O)c1cccc(NC(=O)OC(C)(C)C)c1F ; CC(C)(C)OC(=O)Nc1cccc(C(=O)Cc2ccnc(Cl)n2)c1F]

A solution of Lithium bis(trimethylsilyl)amide (1M in THF, 10.26 mL, 10.26 mmol) was added to a cooled (0° C.) solution of Methyl 3-((tert-butoxycarbonyl)amino)-2-fluorobenzoate (790 mg, 2.93 mmol) in THF (15 mL). After 10 min of stirring, a solution of 2-chloro-4-methylpyrimidine (452 mg, 3.52 mmol) in THF (2 mL) was slowly added and the reaction mixture was allowed to warm to 40° C. for 1 h. Aqueous saturated ammonium chloride was added to the medium. The aqueous layer was extracted with EtOAc (2 times). Combined organics were washed with brine, dried over sodium sulphate, filtered and the filtrate was concentrated under reduced pressure. Purification of the residue by flash chromatography on silica gel (cHex-EtOAc 1/0 to 0/1) afforded the title compound (880 mg, 82%). LC/MS (ES⁺): 366.3-368.3 (M+1).

### tert-butyl (3-(2-(tert-butyl)-5-(2-chloropyrimidin-4-yl)thiazol-4-yl)-2-fluorophenyl)carbamate

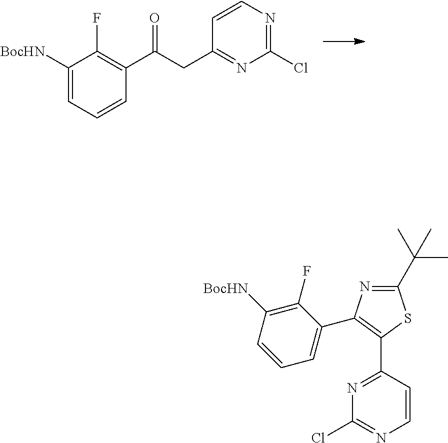 [SMILES: CC(C)(C)OC(=O)Nc1cccc(C(=O)Cc2ccnc(Cl)n2)c1F ; CC(C)(C)OC(=O)Nc1cccc(-c2nc(C(C)(C)C)sc2-c2ccnc(Cl)n2)c1F]

N-Bromosuccinimide (107 mg, 0.60 mmol) was added to a solution of tert-butyl (3-(2-(2-chloropyrimidin-4-yl)acetyl)-2-fluorophenyl)carbamate (200.0 mg, 0.54 mmol) in dimethylacetamide (5.5 mL). The reaction mixture was stirred at room temperature for 1 h then 2,2,2-Trimethylthioacetamide (70 mg, 0.60 mmol) was added. After stirring at room temperature for 1 h, the medium was heated at 50° C. overnight. The mixture was poured onto water and the suspension was filtered. The solid was dried under vacuum to afford the title (220 mg, 86%). LC/MS (ES⁺): 463.3-465.3 (M+1).

### tert-butyl (3-(2-(tert-butyl)-5-(2-(phenylamino)pyrimidin-4-yl)thiazol-4-yl)-2-fluorophenyl)carbamate (Compound 6)

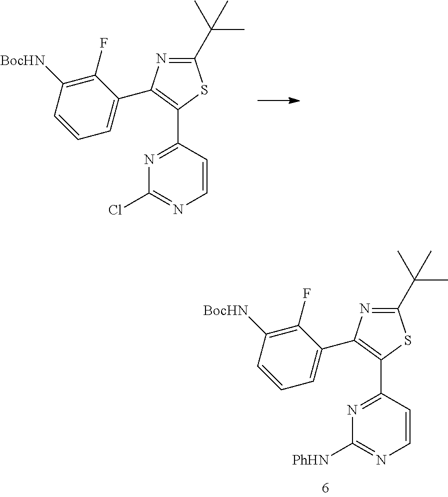

A suspension of tert-butyl (3-(2-(tert-butyl)-5-(2-chloropyrimidin-4-yl)thiazol-4-yl)-2-fluorophenyl)carbamate (77 mg, 0.16 mmol) and aniline (23 mg, 0.25 mmol) in iPrOH (1.6 mL), in presence of catalytic concentrated HCl (4 μL), was stirred at 100° C. for 3 h, under μwaves irradiation. The mixture was concentrated under vacuum. Purification of the residue by preparative TLC (cHex/EtOAc 8/2) afforded the title compound 6 (15 mg, 17%). LC/MS (ES⁺): 520.5 (M+1).

### I.7. Synthesis of Compound 7

### 4-Methyl-7H-pyrrolo[2,3-d]pyrimidine

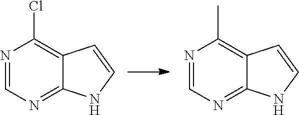

To a previously degassed solution of 4-Chloro-7H-pyrrolo[2,3-d]pyrimidine (418 mg, 2.72 mmol) and palladium tetrakis (157 mg, 0.14 mmol) in THF (27 mL) was added a solution of trimethylaluminium (2.72 mL, 5.44 mmol, 2M in toluene). After refluxing overnight, the mixture was cooled to 0° C. and quenched by slow addition of saturated aqueous ammonium chloride. The medium was diluted with EtOAc. The mixture was filtered and the filtrate was decanted. The organic layer was washed with brine, filtered and concentrated under vacuum to afford impure title compound (338 mg). LC/MS (ES⁺): 134.1 (M+1).

### 4-Methyl-7-((2-(trimethylsilyl)ethoxy)methyl)-7H-pyrrolo[2,3-d]pyrimidine

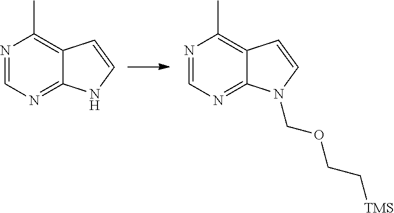

Sodium hydride (36 mg, 0.90 mmol, 60% w/w) was added to a cooled (0° C.) solution of impure 4-Methyl-7H-pyrrolo[2,3-d]pyrimidine (80 mg, 0.6 mmol) in DMF (3 mL). After stirring at 0° C. for 10 min, 2-(trimethylsilyl)ethoxymethyl chloride (120 mg, 0.72 mmol) was added. The mixture was slowly allowed to stir at room temperature for 2 h. The mixture was carefully quenched by addition of saturated aqueous ammonium chloride. The aqueous layer was extracted with EtOAc (three times). The combined organic layer was washed with brine, dried over sodium sulphate, filtered and concentrated under reduced pressure. Purification of the residue by chromatography on silica (cHex/EtOAc) afforded the title compound (85 mg, 53%). LC/MS (ES+): 264.3 (M+1).

### 2,6-difluoro-N-(2-fluoro-3-(2-(7-((2-(trimethylsilyl)ethoxy)methyl)-7H-pyrrolo[2,3-d]pyrimidin-4-yl)acetyl)phenyl)benzenesulfonamide

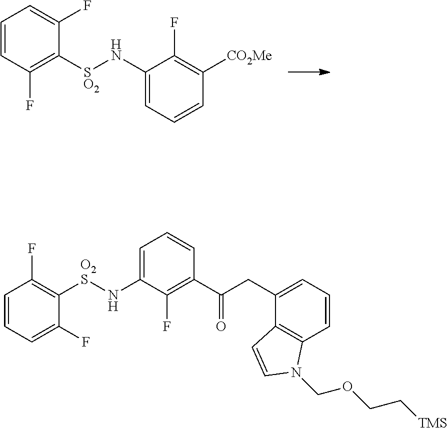 [SMILES: COC(=O)c1cccc(NS(=O)(=O)c2c(F)cccc2F)c1F ; C[Si](C)(C)CCOCn1ccc2c(CC(=O)c3cccc(NS(=O)(=O)c4c(F)cccc4F)c3F)cccc21]

A solution of Lithium bis(trimethylsilyl)amide (1M in THF, 1.01 mL, 1.01 mmol) was added to a cooled (0° C.) solution of Methyl 3-((2,6-difluorophenyl)sulfonamido)-2-fluorobenzoate (100 mg, 0.29 mmol) in THF (2.5 mL). After 10 min of stirring, a solution of 4-Methyl-7-((2-(trimethylsilyl)ethoxy)methyl)-7H-pyrrolo[2,3-d]pyrimidine (76 mg, 0.29 mmol) in THF (1 mL) was slowly added and the reaction mixture was allowed to warm to room temperature. After 2 hours of stirring, saturated aqueous ammonium chloride was added to the medium. The aqueous layer was extracted with EtOAc (two times). Combined organics were washed with brine, dried over sodium sulphate, filtered and the filtrate was concentrated under reduced pressure to afford the title compound (170 mg, quant) as a mixture of ketone and enol. LC/MS (ES⁺): 577.4 (M+1).

### N-(3-(2-(tert-butyl)-5-(7-((2-(trimethylsilyl)ethoxy)methyl)-7H-pyrrolo[2,3-d]pyrimidin-4-yl)thiazol-4-yl)-2-fluorophenyl)-2,6-difluorobenzenesulfonamide

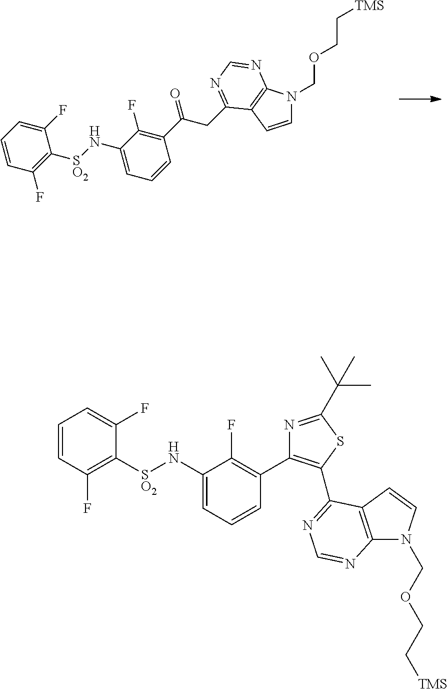 [SMILES: C[Si](C)(C)CCOCn1ccc2c(CC(=O)c3cccc(NS(=O)(=O)c4c(F)cccc4F)c3F)ncnc21 ; CC(C)(C)c1nc(-c2cccc(NS(=O)(=O)c3c(F)cccc3F)c2F)c(-c2ncnc3c2ccn3COCC[Si](C)(C)C)s1]

N-chlorosuccinimide (11.5 mg, 0.086 mmol) was added to a solution of 2,6-difluoro-N-(2-fluoro-3-(2-(7-((2-(trimethylsilyl)ethoxy)methyl)-7H-pyrrolo[2,3-d]pyrimidin-4-yl)acetyl)phenyl)benzenesulfonamide (50 mg, 0.086 mmol) in dimethylacetamide (1 mL). The reaction mixture was stirred at rt for 1 h then 2,2,2-Trimethylthioacetamide (10 mg, 0.086 mmol) was added. The medium was heated at 65° C. overnight. The mixture was then partitioned between water and EtOAc. The aqueous layer was extracted with EtOAc. Combined organics were washed with water, brine, dried over sodium sulphate, filtered and concentrated under vacuum. Purification by preparative TLC (cHex/EtOAc 7/3) afforded the title compound (8 mg, 13%). LC/MS (ES⁺): 674.6 (M+1).

### N-(3-(2-(tert-butyl)-5-(7H-pyrrolo[2,3-d]pyrimidin-4-yl)thiazol-4-yl)-2-fluorophenyl)-2,6-difluorobenzenesulfonamide

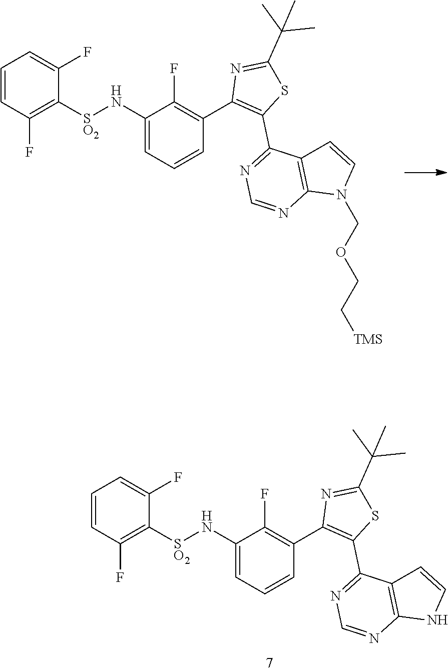 [SMILES: CC(C)(C)c1nc(-c2cccc(NS(=O)(=O)c3c(F)cccc3F)c2F)c(-c2ncnc3c2ccn3COCC[Si](C)(C)C)s1 ; CC(C)(C)c1nc(-c2cccc(NS(=O)(=O)c3c(F)cccc3F)c2F)c(-c2ncnc3[nH]ccc23)s1]

A solution of N-(3-(2-(tert-butyl)-5-(7-((2-(trimethylsilyl)ethoxy)methyl)-7H-pyrrolo[2,3-d]pyrimidin-4-yl)thiazol-4-yl)-2-fluorophenyl)-2,6-difluorobenzenesulfonamide (8 mg, 0.12 mmol) in TFA (0.25 mL) was stirred at room temperature for 30 minutes. The mixture was concentrated under vacuum and the residue was dissolved in MeOH (0.5 mL). A solution of ammonia in methanol (50 μL, 7N) was added to the solution. After 30 minutes of stirring at room temperature, the mixture was concentrated under vacuum. The residue was dissolved in EtOAc and washed with water. The organic layer was dried over sodium sulphate, filtered and the filtrate was concentrated under vacuum. Trituration of the residue in cHex/DCM (9/1) afforded the title compound (3.6 mg, 55%). ¹H NMR (CDCl₃): 9.12 (bs, 1H); 8.76 (s, 1H); 7.69-7.66 (m, 1H); 7.62-7.58 (bs, 1H); 7.57-7.55 (m, 1H); 7.52-7.46 (m, 1H); 7.21 (t, 1H, J=7.9 Hz); 7.09 (bs, 1H); 6.96 (t, 2H, J=8.7 Hz); 5.76 (bs, 1H); 1.51 (s, 9H). LC/MS (ES⁺): 544.4 (M+1).

### I.8. Synthesis of Compound 8

### 2,6-difluoro-N-(2-fluoro-3-(2-(2-hydroxyethyl)-5-(2-(phenylamino)pyrimidin-4-yl)thiazol-4-yl)phenyl)benzenesulfonamide (Compound 8)

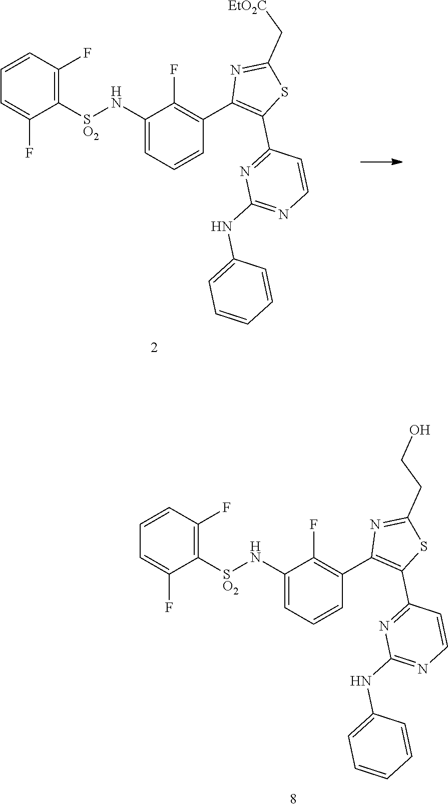 [SMILES: CCOC(=O)Cc1nc(-c2cccc(NS(=O)(=O)c3c(F)cccc3F)c2F)c(-c2ccnc(Nc3ccccc3)n2)s1 ; O=S(=O)(Nc1cccc(-c2nc(CCO)sc2-c2ccnc(Nc3ccccc3)n2)c1F)c1c(F)cccc1F]

A LAH solution (2N in THF, 240 μL, 0.48 mmol) was added drop wise to a cooled (0° C.) solution of Compound 2 (ethyl 2-(4-(3-((2,6-difluorophenyl)sulfonamido)-2-fluorophenyl)-5-(2-(phenylamino)pyrimidin-4-yl)thiazol-2-yl)acetate) (100 mg, 0.16 mmol) in THF (2.5 mL). After 4 h of stirring at room temperature, three more equivalents of LAH (2N in THF, 240 μL, 0.48 mmol) were added and the solution was stirred at room temperature for an additional 2 h. The reaction was quenched by adding at 0° C. aqueous HCl (1N). The mixture was partitioned between EtOAc and water. The aqueous layer was extracted with EtOAc (two times), dried over sodium sulphate and concentrated under reduced pressure. Purification of the residue by two successive preparative TLC (DCM/MeOH 96/4) afforded the title compound 8 (3.7 mg). LC/MS (ES⁺): 584.5 (M+1).

### I.9. Synthesis of Compound 9

### 2-(4-(3-((2,6-difluorophenyl)sulfonamido)-2-fluorophenyl)-5-(2-(phenylamino)pyrimidin-4-yl)thiazol-2-yl)-N-methylacetamide (Compound 9)

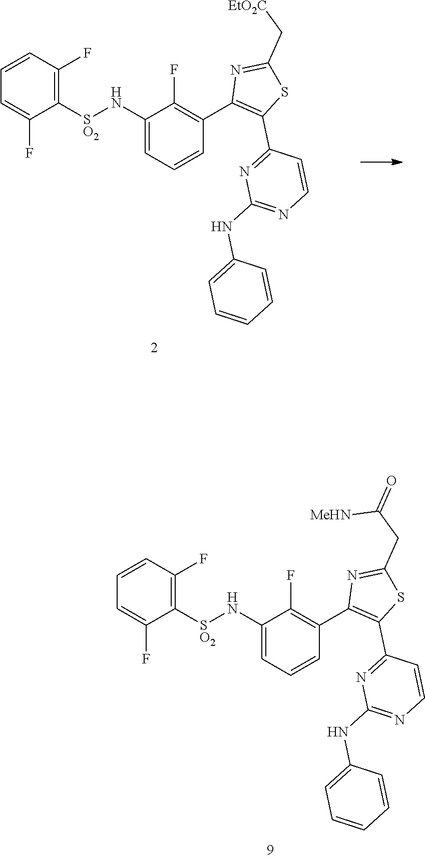 [SMILES: CCOC(=O)Cc1nc(-c2cccc(NS(=O)(=O)c3c(F)cccc3F)c2F)c(-c2ccnc(Nc3ccccc3)n2)s1 ; CNC(=O)Cc1nc(-c2cccc(NS(=O)(=O)c3c(F)cccc3F)c2F)c(-c2ccnc(Nc3ccccc3)n2)s1]

A solution of Compound 2 (ethyl 2-(4-(3-((2,6-difluorophenyl)sulfonamido)-2-fluorophenyl)-5-(2-(phenylamino)pyrimidin-4-yl)thiazol-2-yl)acetate) (100 mg, 0.16 mmol) in MeOH (1.6 mL) was treated with a methylamine solution (9.8N in MeOH, 325 μL, 3.2 mmol). After 30 minutes of stirring at room temperature, the mixture was concentrated under vacuum. The residue was dissolved in EtOAc and the resulting organic layer was washed with saturated aqueous ammonium chloride, with brine, dried over sodium sulphate and concentrated under vacuum. Purification of the residue by flash chromatography on silica gel (DCM-MeOH 1/0 to 95/5) afforded the title compound 9 (60 mg, 58%). LC/MS (ES⁺): 611.5 (M+1).

### I.10. Synthesis of Compound 10

### N-(3-(2-(tert-butyl)-5-(2-(phenylamino)pyrimidin-4-yl)thiazol-4-yl)-2-fluorophenyl)cyclopropanesulfonamide (Compound 10)

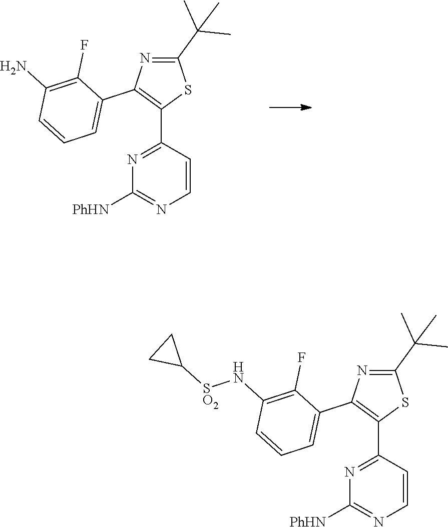

Cyclopropyl sulfonyl chloride (10 μL, 95 μmol) was added to a solution of 4-(4-(3-amino-2-fluorophenyl)-2-(tert-butyl)thiazol-5-yl)-N-phenylpyrimidin-2-amine (20 mg, 47 μmol) in pyridine (1 mL). After 2 d of stirring at room temperature, Cyclopropyl sulfonyl chloride (10 μL, 95 μmol) was added. The mixture was stirred for two additional days. The mixture was concentrated under vacuum and the residue was partitioned between EtOAc and saturated aqueous ammonium chloride. The organic layer was washed with brine, dried over sodium sulphate, filtrated and dried under vacuum. Purification of the residue by preparative TLC (DCM/MeOH 95/5) afforded the title compound 10 (9 mg, 36%). LC/MS (ES⁺): 524.5 (M+1).

### II. Biological Examples

### II.1. LIMK1 Kinase Assay

Materials and Methods

Compounds were tested in a LIMK1 assay performed by Eurofins Panlabs Inc. LIMK1 (h) is incubated with 8 mM MOPS pH 7.0, 0.2 mM EDTA, 0.6 mg/mL cofilin, 10 mM Magnesium acetate and [gamma-33P]-ATP. The reaction is initiated by the addition of the Mg/ATP mix. After incubation for 40 min at room temperature, the reaction is stopped by the addition of phosphoric acid to a concentration of 0.5%. 10 μL of the reaction is then spotted onto a P30 filtermat and washed four times for 4 min in 0.425% phosphoric acid and once in methanol prior to drying and scintillation counting. Compounds were tested at 10; 3; 1; 0.3; 0.1; 0.03; 0.01; 0.003; 0.001 μM and 15 μM ATP. IC₅₀ LIMK1 is then determined.

Results

Results are presented in Table 2 below and are represented as follows: “+” means 500 nM≤IC₅₀<5 000 nM; “++” means 100 nM≤IC₅₀<500 nM; “+++” means 10 nM<IC₅₀<100 nM; “++++” means IC₅₀<10 nM.

TABLE 2

| Compound | IC₅₀ LIMK1 |
|---|---|
| 1 | +++ |
| 2 | ++++ |
| 3 | ++ |
| 4 | ++ |
| 5 | ++ |
| 6 | + |
| 7 | + |
| 8 | ++++ |
| 9 | ++++ |
| 10 | ++ |

### II.2. LIMK2 Kinase Assay

Materials and Methods

LIMK2 Kinase Assay (Compound 1)

Compounds were tested in a LIMK2 assay performed in a final volume of 12.5 μl containing 1.5 μl of compound or equivalent amount of DMSO as control, 1 μl (6.25 ng) of N-terminal 6His-tagged recombinant human LIMK2 fragment 1-638 (Carna Biosciences #09-106, in 20 mM MOPS pH=7.0, 1 mM EDTA, 0.01% NP-40, 5% glycerol, 0.1% 2-mercaptoethanol, 1 mg·mL⁻¹ Bovine Serum Albumin) and a mixture containing 8 mM MOPS pH=7.0, 200 μM EDTA, 110 μM of recombinant GST-cofilin 1, 10 mM MgCl₂, and 360 μM ATP. Assays were performed at 30° C. for 10 min before termination by the addition of 40 μl of Laemmli buffer. Samples are then diluted into 1500 μL H₂O and 100 μL are then diluted with 200 μL TBS. 5 μL are then spotted on a PVDF membrane (Merck-Millipore Immobilon P IPVH00010). After 20 min incubation RT, membrane was blocked with TBS, 0.1% Tween 20 and 5% BSA for 1 h RT under agitation. The membrane was rinsed 3 times 10 min RT under agitation with TBS, 0.1% Tween-20 (TBST) and then 1 h RT with anti-phospho-Ser3-cofilin (Cell Signaling Technology #3313, 1/1000 dilution) antibody diluted in TBS, 0.1% Tween 20 and 1% BSA. The membrane is then rinsed 3 times 10 min RT under agitation in TBST and incubated for 1 hour with anti-rabbit secondary antibody, horseradish peroxidase conjugated (Jackson Immunoresearch #711-036-152) under agitation at room temperature. After three washes 10 min RT in TBST, the detection of phosphorylated cofilin was performed using chemiluminescence kit ECL™ Plus (GE Healthcare RPN2132). IC₅₀ LIMK2 is then determined.

LIMK2 Kinase Assay (Compound 9)

LIMK2(h) is incubated with 8 mM MOPS pH 7.0, 0.2 mM EDTA, 0.63 mg/mL cofilin, 10 mM Magnesium acetate and [9-33P-ATP] (specific activity and concentration as required). The reaction is initiated by the addition of the Mg/ATP mix. After incubation for 120 minutes at room temperature, the reaction is stopped by the addition of phosphoric acid to a concentration of 0.5%. 10 l of the stopped reaction is spotted onto a P30 filtermat and washed four times for 4 minutes in 0.425% phosphoric acid and once in methanol prior to drying and scintillation counting. Compounds were tested at 10; 3; 1; 0.3; 0.1; 0.03; 0.01; 0.003; 0.001 μM and 15 μM ATP. IC₅₀ LIMK2 is then determined.

Results

Results are presented in Table 3 below and are represented as follows: “+” means 500 nM≤IC₅₀<5 000 nM; “++” means 100 nM≤IC₅₀<500 nM; “+++” means 10 nM<IC₅₀<100 nM; “++++” means IC₅₀<10 nM.

TABLE 3

| Compound | IC₅₀ LIMK2 |
|---|---|
| 1 | ++ |
| 9 | ++++ |

### II.3. Kinase Selectivity Analysis

Materials and Methods

Kinase selectivity was performed on a panel of 58 recombinant protein kinases. The assays were performed in the presence of 5 μM inhibitor at the respective Km ATP for each kinase, using the KinaseProfiler panel service (Merck-Millipore). Residual activity measured in the presence of 1 μM inhibitor is expressed as the percent of activity determined in the absence of inhibitor.

Results

Results for compound 1 are presented in Table 4 below and are represented as follows: “−” means 50%≤residual activity; “+” means 10%≤residual activity <50%; “++” means 3%≤residual activity <10% nM; “+++” means residual activity <3%.

TABLE 4

| Kinase | % activity |
|---|---|
| Abl(h) | − |
| ALK(h) | − |
| AMPKα1(h) | − |
| A-Raf(h) | − |
| Aurora-A(h) | − |
| Aurora-B(h) | + |
| BTK(h) | + |
| B-Raf(h) | ++ |
| B-Raf(V599E)(h) | ++ |
| CaMKIβ(h) | − |
| CaMKIIδ(h) | − |
| cKit(h) | − |
| cKit(D816V)(h) | − |
| cKit(D816H)(h) | + |
| cKit(V560G)(h) | + |
| c-RAF(h) | ++ |
| EGFR(h) | − |
| FAK(h) | − |
| FGFR1(h) | − |
| Flt1(h) | + |
| Flt3(D835Y)(h) | − |
| Flt3(h) | − |
| Flt4(h) | ++ |
| GSK3β(h) | − |
| IGF-1R(h), activated | − |
| IKKα(h) | − |
| IR(h), activated | − |
| JAK2(h) | − |
| KDR(h) | + |
| LIMK1(h) | +++ |
| LKB1(h) | − |
| MEK1(h) | − |
| MKK4(m) | − |
| MLCK(h) | − |
| MRCKα(h) | − |
| MRCKβ(h) | − |
| p70S6K(h) | − |
| PAK1(h) | − |
| PAK2(h) | − |
| PAK4(h) | − |
| PAK3(h) | − |
| PAK5(h) | − |
| PAK6(h) | − |
| PDGFRα(h) | − |
| PKBα(h) | − |
| PKCα(h) | − |
| Plk1(h) | − |
| Plk3(h) | − |
| Ret(h) | + |
| ROCK-I(h) | − |
| ROCK-II(h) | − |
| SAPK2a(h) | − |
| Src(1-530)(h) | ++ |
| TAK1(h) | − |
| TrkA(h) | + |
| TrkB(h) | − |
| TrkC(h) | − |
| PI3 Kinase (p110a(E542K)/p85a)(h) | − |

For B-Raf, the IC₅₀ was also determined and compared to LIMK1 IC₅₀ reported above (part II.1), in order to quantify the selectivity of the compounds of the invention for LIMK over B-Raf.

Results are presented in Table 5 below and are represented as follows: “+” means 500 nM≤IC₅₀<5 000 nM; “++” means 100 nM≤IC₅₀<500 nM; “+++” means 10 nM≤IC₅₀<100 nM; “++++” means IC₅₀<10 nM:

TABLE 5

| Compound | IC₅₀ LIMK1 | IC₅₀ B-Raf |
|---|---|---|
| 1 | +++ | + |

For compound 1, the selectivity ratio for LIMK1 over B-Raf, calculated by dividing the IC₅₀ B-Raf (nM) by the IC₅₀ LIMK1 (nM), was determined to be of about 20.

### II.4. Cell Proliferation Assay and Western Blot Analysis of Cellular Cofilin Phosphorylation Status

Materials and Methods

Cell Lines and Cell Culture

MV4-11 cell line was originally purchased from the American Type Culture Collection (ATCC). MV4-11 cells are cultured in RPMI 1640 10% (v/v) FBS supplemented with 100 U/mL⁻¹ penicillin, 0.1 mg·mL⁻¹ streptomycin (PAN Biotech P06-07100) and 2 mM Glutamine (Sigma-Aldrich 59202C). Cells were maintained at 37° C. with 5% CO₂.

Cell Proliferation Assay

The assay was performed in 96 wells microplate (Greiner Bioone 655090). MV4-11 cells are seeded at 5,000 cells per well. Compounds (or equivalent amounts of DMSO) are then added and cells are allowed to grow for 48 h. Proliferation is evaluated using PrestoBlue assay according to manufacturer recommendations. Results are expressed as GI₅₀ (Growth Inhibition 50%: concentration at which proliferation is inhibited of 50%) in comparison to DMSO controls.

Western Blot Analysis of Cellular Cofilin Phosphorylation Status

In a 6 wells plate (Falcon 353046), 2·10⁶ cells in 1.5 mL medium are treated for 2 h with compounds. Cells are then collected and cell lysis is performed at 4° C. using RIPA buffer supplemented with protease inhibitors cocktail (Sigma-Aldrich P8340) and phosphatase inhibitors cocktail (Sigma-Aldrich P5726). Following centrifugation 30 min 15000 g 4° C., supernatants are mixed with Laemmli buffer and denaturation is performed by heating 5 min 95° C. 10 μL of samples are subjected to electrophoresis on 15% SDS-PAGE. After electro-transfer (Mini Transblot, Bio-Rad), the PVDF membrane (Merck-Millipore Immobilon P IPVH00010) is blocked with Tris Buffered Saline, pH 7.4 (TBS) with 0.1% Tween 20 and 5% BSA for 1 h RT under agitation. The membrane was rinsed 3 times 10 min RT under agitation with TBS, 0.1% Tween-20 (TBST) and then 1 h RT with anti-phospho-Ser3-cofilin (Cell Signaling Technology #3313, 1/1000 dilution) antibody or anti-cofilin (Cell Signaling Technology #3312, 1/1000 dilution) diluted in TBS, 0.1% Tween 20 and 1% BSA. The membrane is then rinsed 3 times 10 min RT under agitation in TBST and incubated for 1 hour with anti-rabbit secondary antibody, horseradish peroxidase conjugated (Jackson Immunoresearch #711-036-152) under agitation at room temperature. After three washes 10 min RT in TBST, the detection of total or phosphorylated cofilin was performed using chemiluminescence kit ECL™ Plus (GE Healthcare RPN2132). For each concentration of compound, ratio between phosphorylated cofilin and total cofilin is determined. This ratio is then expressed as percentage of control ratio (i.e ratio from DMSO treated cells). IC₅₀ is then determined.

Results

Cell proliferation results are presented in Table 6 below and are represented as follows: “+” means 5 μM<GI₅₀; “++” means 1 μM<GI₅₀≤5 μM “+++” means GI₅₀≤1 μM.

TABLE 6

| | Cell proliferation assay: GI₅₀ |
|---|---|
| Compound | (μM) |
| 1 | +++ |
| 8 | +++ |
| 9 | ++ |

Cellular cofilin phosphorylation status results are presented in Table 7 below and are represented as follows: “+” means 1 μM≤IC₅₀; “++” means 0.2 μM≤IC₅₀<1 μM; “+++” means IC₅₀<0.2 μM.

TABLE 7

| | IC₅₀ Phospho-Ser3-Cofilin/Total Cofilin |
|---|---|
| Compound | (μM) |
| 1 | +++ |
| 2 | + |
| 3 | +++ |
| 4 | ++ |
| 6 | + |
| 7 | ++ |

### II.5 In Vitro Selectivity Assays

Materials and Methods

LIMK1 Kinase Assay

Compounds were tested in a LIMK1 assay performed by Eurofins Panlabs Inc. LIMK1 (h) is incubated with 8 mM MOPS pH 7.0, 0.2 mM EDTA, 0.6 mg/mL cofilin, 10 mM Magnesium acetate and [gamma-33P]-ATP. The reaction is initiated by the addition of the Mg/ATP mix. After incubation for 40 min at room temperature, the reaction is stopped by the addition of phosphoric acid to a concentration of 0.5%. 10 μL of the reaction is then spotted onto a P30 filtermat and washed four times for 4 min in 0.425% phosphoric acid and once in methanol prior to drying and scintillation counting. Compounds were tested at 10; 3; 1; 0.3; 0.1; 0.03; 0.01; 0.003; 0.001 μM and 15 μM ATP. IC₅₀ LIMK1 is then determined.

B-Raf Kinase Assay

Compounds were tested in a B-Raf assay performed by Eurofins Panlabs Inc. B-Raf (h) is incubated with 25 mM Tris/HCl pH 7.5, 0.2 mM EGTA, 10 mM DTT, 0.01% Triton X-100, 0.5 mM sodium orthovandate, 0.5 mM 6-glycerophosphate, 1% glycerol, 34 nM unactive MEK1, 69 nM unactive MAPK2, 0.5 mg/mL myelin basic protein, and 10 mM Magnesium acetate and [gamma-33P]-ATP. The reaction is initiated by the addition of the Mg/ATP mix. After incubation for 40 min at room temperature, the reaction is stopped by the addition of phosphoric acid to a concentration of 0.5%. 10 μL of the reaction is then spotted onto a P30 filtermat and washed four times for 4 min in 0.425% phosphoric acid and once in methanol prior to drying and scintillation counting. Compounds were tested at 10; 3; 1; 0.3; 0.1; 0.03; 0.01; 0.003; 0.001 μM and 120 μM ATP. IC₅₀ B-Raf is then determined.

Selectivity ratios are calculated by dividing the IC₅₀ B-Raf (nM) by the IC₅₀ LIMK1 (nM).

Results

Results are presented in Table 8 below and are represented as follows: “+” means 500 nM≤IC50<5 000 nM; “++” means 100 nM≤IC50<500 nM; “+++” means 10 nM<IC50<100 nM; “++++” means IC50<10 nM.

Compounds 8 (Na) and 9 (Na) refer respectively to sodium salts of compounds 8 and 9.

TABLE 8

| | IC₅₀ LIMK1 | IC₅₀ B-Raf | Selectivity |
|---|---|---|---|
| Compound | (nM) | (nM) | ratio |
| 1 | +++ | +++ | 8 |
| 2 | ++++ | +++ | 4 |
| 5 | + | + | 11 |
| 6 | + | + | 16 |
| 8 (Na) | ++++ | +++ | 9 |
| 9 | ++++ | +++ | 20 |
| 9 (Na) | ++++ | +++ | 15 |

### II.6. Cellular Selectivity Assays

Materials and Methods

HL-60

Cell Lines and Cell Culture

HL-60 cell line was originally purchased from the Leibniz-Institut Deutsche Sammlung von Mikroorganismen und Zellkulturen GmbH (DSMZ). HL-60 cells are cultured in RPMI 1640 10% (v/v) FBS supplemented with 100 U/mL⁻¹ penicillin, 0.1 mg·mL⁻¹ streptomycin (PAN Biotech P06-07100). Cells were maintained at 37° C. with 5% CO₂.

Cell Proliferation Assay

The assay was performed in 96 wells microplate (Greiner Bioone 655090). HL-60 cells are seeded at 6250 cells per well. Compounds (or equivalent amounts of DMSO) are then added and cells are allowed to grow for 48 h. Proliferation is evaluated using PrestoBlue assay according to manufacturer recommendations. Results are expressed as GI₅₀ (Growth Inhibition 50%: concentration at which proliferation is inhibited of 50%) in comparison to DMSO controls.

K-562

Cell Lines and Cell Culture

K-562 cell line was originally purchased from the Leibniz-Institut Deutsche Sammlung von Mikroorganismen und Zellkulturen GmbH (DSMZ). K-562 cells are cultured in RPMI 1640 10% (v/v) FBS supplemented with 100 U/mL⁻¹ penicillin, 0.1 mg·mL⁻¹ streptomycin (PAN Biotech P06-07100). Cells were maintained at 37° C. with 5% CO₂.

Cell Proliferation Assay

The assay was performed in 96 wells microplate (Greiner Bioone 655090). K-562 cells are seeded at 1562 cells per well. Compounds (or equivalent amounts of DMSO) are then added and cells are allowed to grow for 48 h. Proliferation is evaluated using PrestoBlue assay according to manufacturer recommendations. Results are expressed as GI₅₀ (Growth Inhibition 50%: concentration at which proliferation is inhibited of 50%) in comparison to DMSO controls.

Kasumi-1

Cell Lines and Cell Culture

Kasumi-1 cell line was originally purchased from the American Type Culture Collection (ATCC). Kasumi-1 cells are cultured in RPMI 1640 20% (v/v) FBS supplemented with 100 U/mL⁻¹ penicillin, 0.1 mg·mL⁻¹ streptomycin (PAN Biotech P06-07100). Cells were maintained at 37° C. with 5% CO₂.

Cell Proliferation Assay

The assay was performed in 96 wells microplate (Greiner Bioone 655090). Kasumi-1 cells are seeded at 20000 cells per well. Compounds (or equivalent amounts of DMSO) are then added and cells are allowed to grow for 48 h. Proliferation is evaluated using PrestoBlue assay according to manufacturer recommendations. Results are expressed as GI₅₀ (Growth Inhibition 50%: concentration at which proliferation is inhibited of 50%) in comparison to DMSO controls.

MOLM-13

Cell Lines and Cell Culture

MOLM-13 cell line was originally purchased from the Leibniz-Institut Deutsche Sammlung von Mikroorganismen und Zellkulturen GmbH (DSMZ). MOLM-13 cells are cultured in RPMI 1640 20% (v/v) FBS supplemented with 100 U/mL⁻¹ penicillin, 0.1 mg·mL⁻¹ streptomycin (PAN Biotech P06-07100). Cells were maintained at 37° C. with 5% CO₂.

Cell Proliferation Assay

The assay was performed in 96 wells microplate (Greiner Bioone 655090). MOLM-13 cells are seeded at 1562 cells per well. Compounds (or equivalent amounts of DMSO) are then added and cells are allowed to grow for 48 h. Proliferation is evaluated using PrestoBlue assay according to manufacturer recommendations. Results are expressed as GI₅₀ (Growth Inhibition 50%: concentration at which proliferation is inhibited of 50%) in comparison to DMSO controls.

MOLM-14

Cell Lines and Cell Culture

MOLM-14 cell line was originally purchased from the American Type Culture Collection (ATCC). MOLM-14 cells are cultured in MEM alpha 10% (v/v) FBS supplemented with 100 U/mL⁻¹ penicillin, 0.1 mg·mL⁻¹ streptomycin (PAN Biotech P06-07100). Cells were maintained at 37° C. with 5% CO₂.

Cell Proliferation Assay

The assay was performed in 96 wells microplate (Greiner Bioone 655090). MOLM-14 cells are seeded at 3000 cells per well. Compounds (or equivalent amounts of DMSO) are then added and cells are allowed to grow for 48 h. Proliferation is evaluated using PrestoBlue assay according to manufacturer recommendations. Results are expressed as GI₅₀ (Growth Inhibition 50%: concentration at which proliferation is inhibited of 50%) in comparison to DMSO controls.

MV4-11

Cell Lines and Cell Culture

MV4-11 cell line was originally purchased from the American Type Culture Collection (ATCC) or from the Leibniz-Institut Deutsche Sammlung von Mikroorganismen und Zellkulturen GmbH (DSMZ). MV4-11 cells are cultured in RPMI 1640 20% (v/v) FBS supplemented with 100 U/mL⁻¹ penicillin, 0.1 mg·mL⁻¹ streptomycin (PAN Biotech P06-07100) and 2 mM Glutamine (Sigma-Aldrich 59202C). Cells were maintained at 37° C. with 5% CO₂.

Cell Proliferation Assay

The assay was performed in 96 wells microplate (Greiner Bioone 655090). MV4-11 cells are seeded at 5,000 cells per well. Compounds (or equivalent amounts of DMSO) are then added and cells are allowed to grow for 48 h. Proliferation is evaluated using PrestoBlue assay according to manufacturer recommendations. Results are expressed as GI₅₀ (Growth Inhibition 50%: concentration at which proliferation is inhibited of 50%) in comparison to DMSO controls.

THP-1

Cell Lines and Cell Culture

THP-1 cell line was originally purchased from the Leibniz-Institut Deutsche Sammlung von Mikroorganismen und Zellkulturen GmbH (DSMZ). THP-1 cells are cultured in RPMI 1640 20% (v/v) FBS supplemented with 100 U/mL⁻¹ penicillin, 0.1 mg·mL⁻¹ streptomycin (PAN Biotech P06-07100). Cells were maintained at 37° C. with 5% CO₂.

Cell Proliferation Assay

The assay was performed in 96 wells microplate (Greiner Bioone 655090). THP-1 cells are seeded at 5,000 cells per well. Compounds (or equivalent amounts of DMSO) are then added and cells are allowed to grow for 48 h. Proliferation is evaluated using PrestoBlue assay according to manufacturer recommendations. Results are expressed as GI₅₀ (Growth Inhibition 50%: concentration at which proliferation is inhibited of 50%) in comparison to DMSO controls.

Western Blot Analysis (for all Cell Lines)

Western Blot Analysis of Cellular Cofilin Phosphorylation Status

In a 6 wells plate (Falcon 353046), 2·10⁶ cells in 1.5 mL medium are treated for 2 h with compounds. Cells are then collected and cell lysis is performed at 4° C. using RIPA buffer supplemented with protease inhibitors cocktail (Sigma-Aldrich P8340) and phosphatase inhibitors cocktail (Sigma-Aldrich P5726). Following centrifugation 30 min 15000 g 4° C., supernatants are mixed with Laemmli buffer and denaturation is performed by heating 5 min 95° C. 10 μL of samples are subjected to electrophoresis on 15% SDS-PAGE. After electro-transfer (Mini Transblot, Bio-Rad), the PVDF membrane (Merck-Millipore Immobilon P IPVH00010) is blocked with Tris Buffered Saline, pH 7.4 (TBS) with 0.1% Tween 20 and 5% BSA for 1 h RT under agitation. The membrane was rinsed 3 times 10 min at room temperature under agitation with TBS, 0.1% Tween-20 (TBST) and then 1 h at room temperature with anti-phospho-Ser3-cofilin (Cell Signaling Technology #3313, 1/1000 dilution) antibody or anti-cofilin (Cell Signaling Technology #3312, 1/1000 dilution) diluted in TBS, 0.1% Tween 20 and 1% BSA. The membrane is then rinsed 3 times 10 min at room temperature under agitation in TBST and incubated for 1 h with anti-rabbit secondary antibody, horseradish peroxidase conjugated (Jackson Immunoresearch #711-036-152) under agitation at room temperature. After three washes 10 min at room temperature in TBST, the detection of total or phosphorylated cofilin was performed using chemiluminescence kit ECL™ Plus (GE Healthcare RPN2132). For each concentration of compound, ratio between phosphorylated cofilin and total cofilin is determined. This ratio is then expressed as percentage of control ratio (i.e., ratio from DMSO treated cells). IC₅₀ Phospho-Ser3-Cofilin/Total Cofilin is then determined.

Western Blot Analysis of Cellular MEK1 Phosphorylation Status

In a 6 wells plate (Falcon 353046), 2·10⁶ cells in 1.5 mL medium are treated for 2 h with compounds. Cells are then collected and cell lysis is performed at 4° C. using RIPA buffer supplemented with protease inhibitors cocktail (Sigma-Aldrich P8340) and phosphatase inhibitors cocktail (Sigma-Aldrich P5726). Following centrifugation 30 min 15000 g 4° C., supernatants are mixed with Laemmli buffer and denaturation is performed by heating 5 min 95° C. 10 μL of samples are subjected to electrophoresis on 15% SDS-PAGE. After electro-transfer (Mini Transblot, Bio-Rad), the PVDF membrane (Merck-Millipore Immobilon P IPVH00010) is blocked with Tris Buffered Saline, pH 7.4 (TBS) with 0.1% Tween 20 and 5% BSA for 1 h at room temperature under agitation. The membrane was rinsed 3 times 10 min at room temperature under agitation with TBS, 0.1% Tween-20 (TBST) and then 1 h at room temperature with anti-phospho-Ser218/222-MEK1 (Merck-Millipore 07-461, 1/1000 dilution) antibody or anti-MEK1 (Merck-Millipore 07-641, 1/1000 dilution) diluted in TBS, 0.1% Tween 20 and 1% BSA. The membrane is then rinsed 3 times 10 min RT under agitation in TBST and incubated for 1 h with anti-rabbit secondary antibody, horseradish peroxidase conjugated (Jackson Immunoresearch #711-036-152) under agitation at room temperature. After three washes 10 min at room temperature in TBST, the detection of total or phosphorylated cofilin was performed using chemiluminescence kit ECL™ Plus (GE Healthcare RPN2132). For each concentration of compound, ratio between phosphorylated cofilin and total cofilin is determined. This ratio is then expressed as percentage of control ratio (i.e., ratio from DMSO treated cells). IC₅₀ P-MEK/Total MEK is then determined.

Selectivity ratios are calculated by dividing the IC₅₀ P-MEK/Total MEK value (μM) by the IC₅₀ Phospho-Ser3-Cofilin/Total Cofilin value (μM).

Results

Results for compound 1 are presented in Table 9 below and are represented as follows: “+” means 1 μM≤IC₅₀; “++” means 0.2 μM≤IC₅₀<1 μM; “+++” means IC₅₀<0.2 μM.

TABLE 9

| | IC₅₀ Phospho-Ser3-Cofilin/ | IC₅₀ P-MEK/ | |
|---|---|---|---|
| | Total Cofilin | Total MEK | Selectivity |
| Cell line | (μM) | (μM) | ratio |
| MV4-11 | +++ | + | 125 |
| K562 | ++ | + | 8 |
| MOLM-14 | +++ | + | 40 |

Results for compound 8 are presented in Table 10 below and are represented as follows: “+” means 1 μM≤IC₅₀; “++” means 0.2 μM≤IC₅₀<1 μM; “+++” means IC₅₀<0.2 μM.

TABLE 10

| | IC₅₀ Phospho-Ser3-Cofilin/ | IC₅₀ P-MEK/ | |
|---|---|---|---|
| | Total Cofilin | Total MEK | Selectivity |
| Cell line | (μM) | (μM) | ratio |
| THP-1 | +++ | ++ | 80 |
| MV4-11 | ++ | + | 4 |
| HL-60 | +++ | + | 31 |
| K562 | +++ | ++ | 20 |
| MOLM-14 | +++ | + | 100 |

Results for compound 8 (Na), i.e., sodium (Na) salt of compound 8, are presented in Table 11 below and are represented as follows: “+” means 1 μM≤IC₅₀; “++” means 0.2 μM≤IC₅₀<1 μM; “+++” means IC₅₀<0.2 μM.

TABLE 11

| | IC₅₀ Phospho-Ser3-Cofilin/ | IC₅₀ P-MEK/ | |
|---|---|---|---|
| | Total Cofilin | Total MEK | Selectivity |
| Cell line | (μM) | (μM) | ratio |
| Kasumi-1 | +++ | + | 10 |
| MV4-11 | +++ | + | 6 |
| HL-60 | +++ | + | 42 |
| MOLM-13 | +++ | + | 42 |
| MOLM-14 | +++ | + | 30 |

Results for compound 9 are presented in Table 12 below and are represented as follows: “+” means 1 μM≤IC₅₀; “++” means 0.2 μM≤IC₅₀<1 μM; “+++” means IC₅₀<0.2 μM.

TABLE 12

| | IC₅₀ Phospho-Ser3-Cofilin/ | IC₅₀ P-MEK/ | |
|---|---|---|---|
| | Total Cofilin | Total MEK | Selectivity |
| Cell line | (μM) | (μM) | ratio |
| THP-1 | +++ | ++ | 8 |
| MV4-11 | ++ | + | 3 |
| K562 | +++ | + | 7 |
| MOLM-13 | +++ | + | 125 |
| MOLM-14 | +++ | + | 125 |

Results for compound 9 (Na), i.e., sodium (Na) salt of compound 9, are presented in Table 13 below and are represented as follows: “+” means 1 μM≤IC₅₀; “++” means 0.2 μM≤IC₅₀<1 μM; “+++” means IC₅₀<0.2 μM.

TABLE 13

| | IC₅₀ Phospho-Ser3-Cofilin/ | IC₅₀ P-MEK/ | |
|---|---|---|---|
| | Total Cofilin | Total MEK | Selectivity |
| Cell line | (μM) | (μM) | ratio |
| Kasumi-1 | ++ | + | 25 |
| HL-60 | ++ | + | 15 |
| MOLM-13 | ++ | + | 42 |
| MOLM-14 | +++ | ++ | 90 |

1. A compound of Formula I: 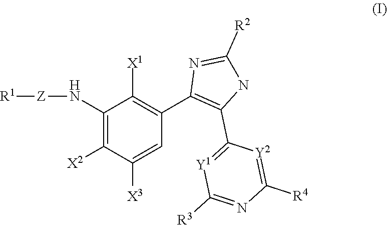
or a pharmaceutically acceptable salt or solvate thereof, wherein
X¹, X² and X³ represent each independently H, halo or cyano, with the condition that at least one of X¹, X² and X³ represents halo or cyano;
Z represents a single bound, —SO₂—, —CO—CO—, —O—CR^{1′}R^{1″}—CO—, —O—CO—, oxazolyl or oxadiazolyl;
R¹ represents H, alkyl, haloalkyl, cycloalkyl, oxacycloalkyl, aryl, arylalkyl or heteroaryl, wherein alkyl, aryl, arylalkyl and heteroaryl groups are optionally substituted by one or more group selected from halo, alkyl, haloalkyl and alkoxy;
R^{1′} and R^{1″} represent each independently halo, alkyl, alkoxyalkyl, aryl or heteroaryl, wherein aryl and heteroaryl groups are optionally substituted by one or more group selected from halo, alkyl, haloalkyl and alkoxy;
R² represents H, alkyl, hydroxyalkyl, alkoxyalkyl, alkyloxycarbonylalkyl, alkylaminocarbonylalkyl or aminocarbonylalkyl;
Y¹ represents N or CH;
Y² represents N or CR⁵;
R³ represents H or NHR⁶;
R⁴ represents H, NR⁷R⁸ or R⁴ is linked with R⁵ when Y² represents CR⁵;
R⁵ represents H or R⁵ is linked with R⁴;
wherein when R⁴ and R⁵ are linked together, —R⁴—R⁵— represents —NH—CH═CR⁹—;
R⁶ represents H or aryl;
R⁷ and R⁸ represent each independently H, aryl, alkyl, hydroxyalkyl, alkoxyalkyl, aminoalkyl, morpholinyl or piperazinyl; and
R⁹ represents H, halo or alkyl;
provided that R³ and R⁴ are not both H;
provided that when Y¹ is CH and R³ is H, then Y² is not N;
provided that when Y¹ is N, Y² is CH, R³ is NHR⁶ and R⁴ is H, then R⁶ is not H; and
provided that compound of Formula I is not N-(3-(2-(tert-butyl)-5-(1H-pyrrolo[2,3-b]pyridin-4-yl)thiazol-4-yl)-2-fluorophenyl)-2,6-difluorobenzenesulfonamide.

2. The compound according to claim 1, having Formula Ia, Ib, Ic, Id or Ie: 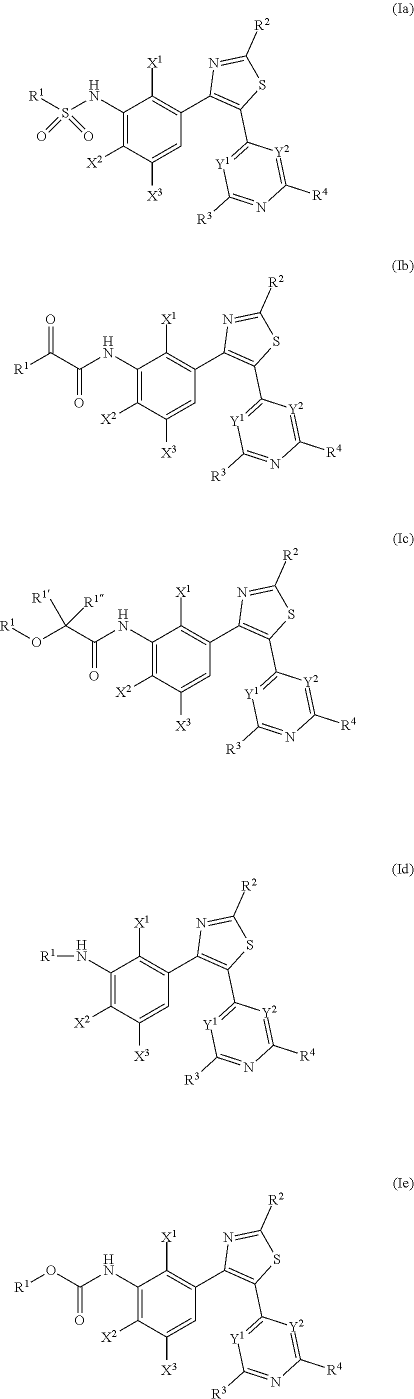
or a pharmaceutically acceptable salt or solvate thereof.

3. The compound according to claim 1, having Formula Ia-U0, Ia-U1a, Ia-U1b, Ia-U3a, Ia-U3b or Ia-U8: 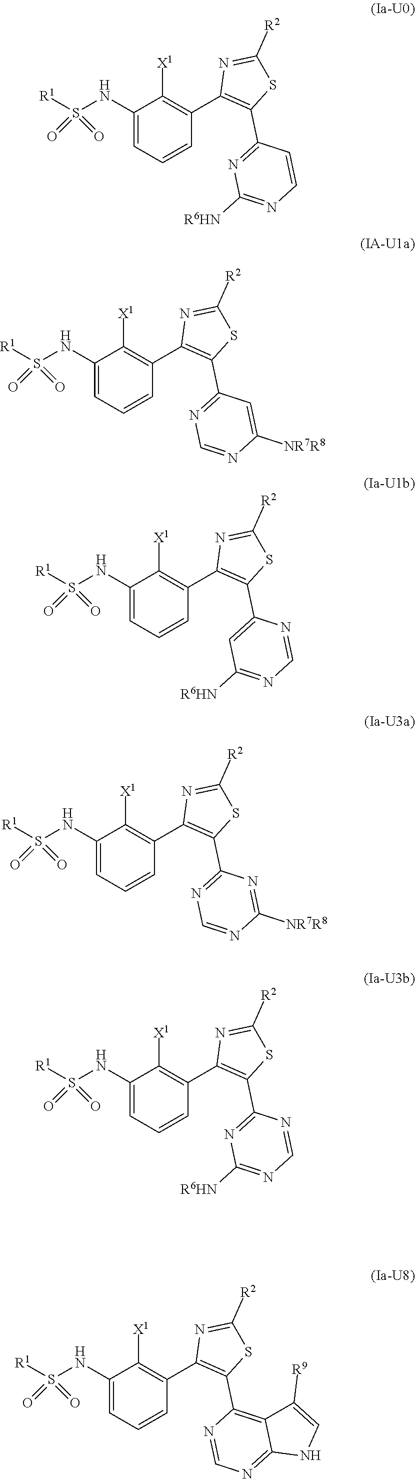
or a pharmaceutically acceptable salt or solvate thereof.

4. The compound according to claim 1, having Formula Ia-U0-1′: 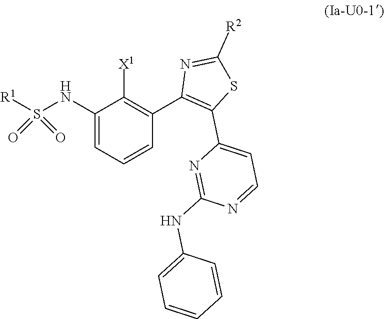
or a pharmaceutically acceptable salt or solvate thereof.

5. The compound according to claim 1, selected from the group consisting of:
N-(3-(2-(tert-butyl)-5-(2-(phenylamino)pyrimidin-4-yl)thiazol-4-yl)-2-fluorophenyl)-2,6-difluorobenzenesulfonamide;
ethyl 2-(4-(3-(2,6-difluorophenylsulfonamido)-2-fluorophenyl)-5-(2-(phenylamino)pyrimidin-4-yl)thiazol-2-yl)acetate;
N-(3-(2-(tert-butyl)-5-(2-(phenylamino)pyrimidin-4-yl)thiazol-4-yl)-2-fluorophenyl)propane-1-sulfonamide;
N-(3-(2-(tert-butyl)-5-(2-(phenylamino)pyrimidin-4-yl)thiazol-4-yl)-2-fluorophenyl)-3,3,3-trifluoropropane-1-sulfonamide;
N-(3-(2-(tert-butyl)-5-(2-(phenylamino)pyrimidin-4-yl)thiazol-4-yl)-2-fluorophenyl)-2-(2,6-difluorophenyl)-2-oxoacetamide;
tert-butyl (3-(2-(tert-butyl)-5-(2-(phenylamino)pyrimidin-4-yl)thiazol-4-yl)-2-fluorophenyl)carbamate;
N-(3-(2-(tert-butyl)-5-(7H-pyrrolo[2,3-d]pyrimidin-4-yl)thiazol-4-yl)-2-fluorophenyl)-2,6-difluorobenzenesulfonamide;
2,6-difluoro-N-(2-fluoro-3-(2-(2-hydroxyethyl)-5-(2-(phenylamino)pyrimidin-4-yl)thiazol-4-yl)phenyl)benzenesulfonamide;
2-(4-(3-(2,6-difluorophenylsulfonamido)-2-fluorophenyl)-5-(2-(phenylamino)pyrimidin-4-yl)thiazol-2-yl)-N-methylacetamide;
N-(3-(2-(tert-butyl)-5-(2-(phenylamino)pyrimidin-4-yl)thiazol-4-yl)-2-fluorophenyl)cyclopropanesulfonamide;
and pharmaceutically acceptable salts or solvates thereof.

6. A pharmaceutical composition comprising a compound according to claim 1, or a pharmaceutically acceptable salt or solvate thereof, and at least one pharmaceutically acceptable excipient.

7. (canceled)

8. A method for treating and/or preventing a LIMK-related disease, comprising administering to a subject in need thereof a compound according to claim 1, or a pharmaceutically acceptable salt or solvate thereof.

9. The method according to claim 8, wherein the LIMK-related disease is selected from proliferative conditions, neurodegenerative disorders, neurodevelopmental disorders, cardiovascular and vascular diseases, eye diseases, airway diseases, inflammatory diseases, skin diseases, intestinal diseases, kidney diseases, bone diseases, viral diseases, drug addiction and neurofibromatosis.

10. The method according to claim 9, wherein the proliferative conditions are selected from tumors, cancers, neoplasms, hyperplasias, psoriasis, bone diseases, fibroproliferative disorders, pulmonary fibrosis, atherosclerosis and smooth muscle cell proliferation in the blood vessels.

11. The method according to claim 9, wherein the proliferative condition is selected from:
carcinomas;
hematopoietic tumors of lymphoid lineage;
hematopoietic tumors of myeloid lineage;
tumors of mesenchymal origin;
tumors of the central or peripheral nervous system;
melanoma; seminoma; teratocarcinoma; osteosarcoma; xeroderma pigmentosum;
keratoacanthoma; thyroid follicular cancer; and Kaposi's sarcoma.

12. The method according to claim 8, wherein the LIMK-related disease is acute myeloid leukemia.

13. A process of manufacturing a compound according to claim 1, or a pharmaceutically acceptable salt or solvate thereof, comprising the following steps:
a) reacting intermediate (A) 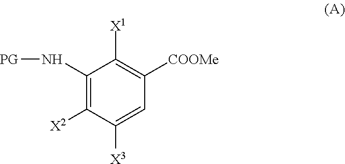
wherein PG represents an amino-protecting group; and wherein X¹, X², and X³ are as defined in claim 1;
with intermediate (B) 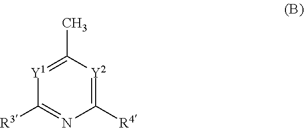
wherein Y¹ and Y² are as defined in claim 1; and
wherein R^{3′} and R^{4′} independently either represent respectively R³ or R⁴ as defined in claim 1, or a precursor of respectively R³ or R⁴;
in presence of a strong base, to afford intermediate (C) 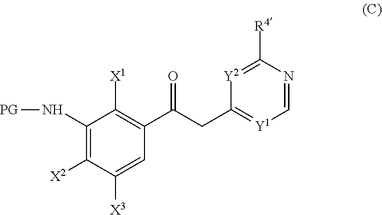
b) forming a thiazole ring by reacting intermediate (C) in presence of N-bromosuccinimide and intermediate (D) 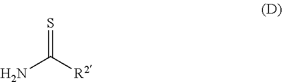
wherein R^{2′} either represents R² as defined in claim 1, or a precursor of R²;
to afford intermediate E 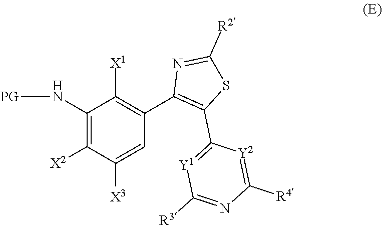
c) deprotecting intermediate (E) in conditions adapted to remove PG, to afford intermediate (F) 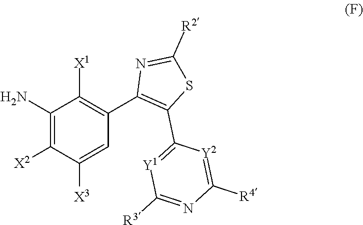
d) introducing R¹ moiety as defined in claim 1 on intermediate (F) by suitable coupling reaction adapted to —Z— linker as defined in claim 1 to afford compound of Formula I′ 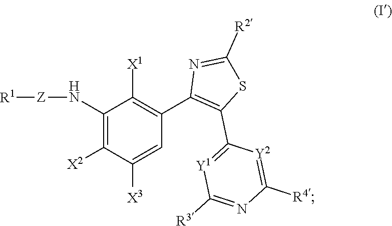
and in case wherein R^{2′}, R^{3′} and/or R^{4′} represent precursors of respectively R², R³ or R⁴ performing one or more additional intermediate steps or final steps of conversion of R^{2′} into R² and/or R^{3′} into R³ and/or of R^{4′} into R⁴.

14. The method according to claim 11, wherein carcinomas are selected from carcinomas of the bladder, breast, colon, bowel, rectum, kidney, epidermal, liver, lung, oesophagus, gall bladder, ovary, uterus, endometrium, pancreas, stomach, cervix, thyroid, prostate, testicle, skin, brain, nerve and bone.

15. The method according to claim 11, wherein hematopoietic tumors of lymphoid lineage are selected from leukemia, acute lymphocytic leukemia, B-cell lymphoma, T-cell lymphoma, Hodgkin's lymphoma, non-Hodgkin's lymphoma, hairy cell lymphoma, and Burkett's lymphoma.

16. The method according to claim 11, wherein tumors of mesenchymal origin are selected from fibrosarcoma and rhabdomyosarcoma.

17. The method according to claim 11, wherein tumors of the central or peripheral nervous system are selected from astrocytoma, neuroblastoma, glioma and schwannoma.
